(19) United States
(12) Patent Application Publication
(10) Pub. No.: US 2020/0046284 A1
(43) Pub. Date: Feb. 13, 2020

(54) SYSTEM, METHOD, AND PROGRAM FOR RECOGNIZING MYOELECTRIC SIGNAL-ORIGINATING MOTION

(71) Applicant: Meltin MMI Co., Ltd., Tokyo (JP)

(72) Inventors: Tatsuya SEKI, Tokyo (JP); Toshiaki ISHII, Tokyo (JP); Hiroyuki YAMADA, Tokyo (JP); Fumiyo TAMURA, Tokyo (JP); Takeshi KIKUTANI, Tokyo (JP)

(21) Appl. No.: 16/486,006

(22) PCT Filed: Jul. 30, 2018
(86) PCT No.: PCT/JP2018/028492

§ 371 Date: Aug. 14, 2019

(30) Foreign Application Priority Data
Jul. 28, 2017 (JP) 2017-147242

(51) Int. Cl. A61B 5/00; A61B 5/0488; A61B 5/11

(52) U.S. Cl. CPC A61B 5/4205; A61B 5/0488; A61B 5/7264; A61B 5/4542; A61B 5/7257; A61B 5/11

(57) ABSTRACT

A system (10) for evaluating a movement of a subject comprises: detection means (111, 112) for detecting a myoelectric signal indicating an activity of a muscle when a subject performs a movement intended to be a predetermined movement comprising a jaw movement, a tongue movement, or a throat movement; processing means (221) for outputting a processed myoelectric signal by applying predetermined processing on the detected myoelectric signal; and determination means (222) for determining a motion level in the predetermined movement of the subject, based on a pattern contained in the processed myoelectric signal.

### TECHNICAL FIELD

The present invention relates to a system, a method, and a program for recognizing a myoelectric signal-originating motion.

### BACKGROUND ART

An oral sensor capable of detecting a motion of tongue movement has been developed (see, for example, Patent Literature 1).

### CITATION LIST

### Patent Literature

[PTL 1] International Publication No. WO 2013/085038

### SUMMARY OF INVENTION

### Technical Problem

Since oral sensors are installed inside the oral cavity to measure the pressure applied by the tongue in the oral cavity, motions of tongue movement that do not involve a contact between the tongue and the sensor, motions of jaw movement or throat movement that are not tongue movement could not be detected.

The objective of the present invention is to provide a system, a method, and a program for recognizing myoelectric signal-originating motions comprising at least two of a motion of a jaw movement, a motion of a tongue movement, and a motion of a throat movement.

Another objective of the present invention is to provide a system, a method, and a program for evaluating a movement of a subject.

### Solution to Problem

The system for recognizing a myoelectric signal-originating motion of the invention comprises detection means for detecting a myoelectric signal indicating activities of a plurality of muscles, processing means for outputting a processed myoelectric signal by applying predetermined processing on the detected myoelectric signal, and determination means for determining which one or more of a plurality of patterns of motions comprising at least two of a pattern of a motion of a jaw movement, a pattern of a motion of a tongue movement, and a pattern of a motion of a throat movement is a pattern contained in the processed myoelectric signal.

In one embodiment, the predetermined processing comprises frequency analysis processing, and the processed myoelectric signal comprises time series data for a frequency distribution of the myoelectric signal.

In one embodiment, the frequency analysis processing comprises a short-time FFT.

In one embodiment, the predetermined processing further comprises signal intensity extraction processing, and the processed myoelectric signal further comprises time series data for signal intensities of the myoelectric signal.

In one embodiment, the determination means comprises learning means for learning at least two of a supervisory pattern of a motion of a jaw movement, a supervisory pattern of a motion of a tongue movement, and a supervisory pattern of a motion of a tongue movement, calculation means for calculating a degree of similarity between a pattern contained in the processed myoelectric signal and the supervisory pattern, and judgment means for judging whether the degree of similarity exceeds a predetermined threshold value.

In one embodiment, the detection means comprises a myoelectric sensor comprising two pairs of measurement electrodes, and the two pairs of measurement electrodes are configured to be able to detect a myoelectric signal indicating activities of at least two of a jaw muscle, a tongue muscle, and a throat muscle.

The method for recognizing a myoelectric signal-originating motion of the invention comprises: detecting a myoelectric signal indicating activities of a plurality of muscles; outputting a processed myoelectric signal by applying predetermined processing on the detected myoelectric signal; and determining which one or more of a plurality of patterns of motions comprising at least two of a pattern of a motion of a jaw movement, a pattern of a motion of a tongue movement, and a pattern of a motion of a throat movement is a pattern contained in the processed myoelectric signal.

The program for recognizing a myoelectric signal-originating motion of the invention is executed in a computer apparatus comprising a processor unit, wherein the program, when executed, makes the processor unit perform processing comprising: receiving a myoelectric signal indicating activities of a plurality of muscles; outputting a processed myoelectric signal by applying predetermined processing on the received myoelectric signal; and determining which one or more of a plurality of patterns of motions comprising at least two of a pattern of a motion of a jaw movement, a pattern of a motion of a tongue movement, and a pattern of a motion of a throat movement is a pattern contained in the processed myoelectric signal.

The present invention provides, for example, the following.

### (Item 1)

A system for evaluating a movement of a subject, the system comprising:

detection means for detecting a myoelectric signal indicating an activity of a muscle when a subject performs a movement intended to be a predetermined movement comprising a jaw movement, a tongue movement, or a throat movement;

processing means for outputting a processed myoelectric signal by applying predetermined processing on the detected myoelectric signal; and

determination means for determining a motion level in the predetermined movement of the subject, based on a pattern contained in the processed myoelectric signal.

### (Item 2)

The system of item 1, wherein the predetermined processing comprises frequency analysis processing, and the processed myoelectric signal comprises time series data for a frequency distribution of the myoelectric signal.

### (Item 3)

The system of item 2, wherein the frequency analysis processing comprises a short-time FFT.

### (Item 4)

The system of item 2 or 3, wherein the predetermined processing further comprises signal intensity extraction processing, and the processed myoelectric signal further comprises time series data for signal intensities of the myoelectric signal.

### (Item 5)

The system of any one of items 1 to 4, wherein the determination means is subjected to learning processing by using a supervisory pattern of a motion corresponding to the predetermined movement among a supervisory pattern of a motion of a jaw movement, a supervisory pattern of a motion of a tongue movement, and a supervisory pattern of a motion of a tongue movement, so that a degree of similarity to a myoelectric signal generated when there is no impairment in the predetermined movement is outputted,

wherein the determination means comprises:

calculation means for calculating a degree of similarity between the pattern contained in the processed myoelectric signal and the supervisory pattern.

### (Item 6)

The system of item 5, wherein the determination means further evaluates the presence or absence of an impairment in the predetermined movement based on the degree of similarity.

### (Item 7)

The system of item 5 or 6, wherein the learning processing is applied using a supervisory pattern of a motion of a jaw movement, a supervisory pattern of a motion of a tongue movement, and a supervisory pattern of a motion of a throat movement.

### (Item 8)

The system of item 7, wherein the learning processing is applied using at least a supervisory pattern of a mouth opening and closing motion associated with the motion of a jaw movement, at least a supervisory pattern of a tongue raising motion, a supervisory pattern of a right side translation motion of the tongue, a supervisory pattern of a left side translation motion of the tongue, and a supervisory pattern of a forward extension motion of the tongue associated with the motion of a tongue movement, and at least a supervisory pattern of a dry swallowing motion associated with the motion of a throat movement.

### (Item 9)

The system of item 8, wherein the learning processing is applied by further using a supervisory pattern of a tapping motion associated with the motion of a jaw movement and a supervisory pattern of a left and right translation motion of the tongue associated with the motion of a tongue movement.

### (Item 10)

The system of any one of items 1 to 9, wherein the detection means comprises a myoelectric sensor comprising two pairs of measurement electrodes, and wherein the two pairs of measurement electrodes are configured to be able to detect a myoelectric signal indicating an activity of a jaw muscle, a tongue muscle, or a throat muscle.

### (Item 11)

A method for evaluating a movement of a subject, the method comprising:

detecting a myoelectric signal indicating an activity of a muscle when a subject performs a movement intended to be a predetermined movement comprising a jaw movement, a tongue movement, or a throat movement;

outputting a processed myoelectric signal by applying predetermined processing on the detected myoelectric signal; and

determining a motion level in the predetermined movement of the subject, based on a pattern contained in the processed myoelectric signal.

### (Item 12)

A program for evaluating a movement of a subject, the program executed in a computer apparatus comprising a processor unit, wherein the program, when executed, causes the processor unit to perform processing comprising:

receiving a myoelectric signal indicating an activity of a muscle when a subject performs a movement intended to be a predetermined movement comprising a jaw movement, a tongue movement, or a throat movement;

outputting a processed myoelectric signal by applying predetermined processing on the received myoelectric signal; and

determining a motion level in the predetermined movement of the subject, based on a pattern contained in the processed myoelectric signal.

### (Item 13)

A classifier used for evaluating a movement of a subject, the classifier comprising:

detection means for detecting a myoelectric signal indicating an activity of a muscle when a subject with no impairment in a predetermined movement comprising a jaw movement, a tongue movement, or a throat movement performs the predetermined movement;

processing means for outputting a processed myoelectric signal by applying predetermined processing on the detected myoelectric signal; and

learning means for learning a pattern contained in the processed myoelectric signal as a pattern in the predetermined movement.

### (Item 14)

The classifier of item 13, wherein the predetermined movement comprises a jaw movement, a tongue movement, and a throat movement.

### (Item 15)

The classifier of item 14, wherein the predetermined movement comprises at least a mouth opening and closing motion associated with a motion of the jaw movement, at least a tongue raising motion, a right side translation motion of the tongue, a left side translation motion of the tongue, and a forward extension motion of the tongue associated with a motion of the tongue movement, and at least a dry swallowing motion associated with a motion of the throat movement.

### (Item 16)

The classifier of item 15, wherein the predetermined movement further comprises a tapping motion associated with a motion of the jaw movement and a left and right translation motion of the tongue associated with a motion of the tongue movement.

### (Item 17)

A method for constructing a classifier used for evaluating a movement of a subject, the method comprising:

detecting a myoelectric signal indicating an activity of a muscle when a subject with no impairment in a predetermined movement comprising a jaw movement, a tongue movement, or a throat movement performs the predetermined movement;

outputting a processed myoelectric signal by applying predetermined processing on the detected myoelectric signal; and learning a pattern contained in the processed myoelectric signal as a pattern in the predetermined movement.

### (Item 18)

A program for constructing a classifier used for evaluating a movement of a subject, the program executed in a computer apparatus comprising a processor unit, wherein the program, when executed, causes the processor unit to perform processing comprising:

receiving a myoelectric signal indicating an activity of a muscle when a subject with no impairment in a predetermined movement comprising a jaw movement, a tongue movement, or a throat movement performs the predetermined movement;

outputting a processed myoelectric signal by applying predetermined processing on the detected myoelectric signal; and

learning a pattern contained in the processed myoelectric signal as a pattern in the predetermined movement.

### Advantageous Effects of Invention

The present invention can provide a system, a method, and a program for recognizing myoelectric signal-originating motions comprising at least two of a motion of a jaw movement, a motion of a tongue movement, and a motion of a throat movement.

The present invention can also provide a system, a method, and a program for evaluating a movement of a subject.

### BRIEF DESCRIPTION OF DRAWINGS

FIG. 1 is a diagram showing a myoelectric device 100 fitted onto a subject.

FIG. 2 (A) is a graph showing an example of an output after the system of the invention has applied predetermined processing on a myoelectric signal detected by the myoelectric device 100 upon a motion of a jaw movement of a subject. (B) is a graph showing an example of an output after the system of the invention has applied predetermined processing on a myoelectric signal detected by the myoelectric device 100 upon a motion of a tongue movement of a subject. (C) is a graph showing an example of an output after the system of the invention has applied predetermined processing on a myoelectric signal detected by the myoelectric device 100 upon a motion of a throat movement of a subject.

FIG. 3 is a block diagram depicting an example of a configuration of system 10 for recognizing a myoelectric signal-originating motion.

FIG. 4 is a block diagram depicting an example of a configuration of a computer apparatus 200.

FIG. 5A is a flow chart depicting an example of processing for recognizing a myoelectric signal-originating motion.

FIG. 5B is a flow chart depicting an example of processing 510 for evaluating a movement of a subject.

FIG. 6-1 (A)-(B) are graphs showing an example of an output after frequency analysis processing by processing means 221 in step S502.

FIG. 6-2 (C)-(F) are graphs showing an example of an output after frequency analysis processing by processing means 221 in step S502.

FIG. 6-3 (G)-(J) are graphs showing an example of an output after frequency analysis processing by processing means 221 in step S502.

FIG. 7A is a flowchart depicting an example of processing in step S503 (processing performed by determination means 222 for determining which one or more of a plurality of patterns of motions comprising at least two of a pattern of a motion of a jaw movement, a pattern of a motion of a tongue movement, and a pattern of a motion of a throat movement is a pattern contained in a processed myoelectric signal).

FIG. 7B is a flowchart depicting an example of processing in step S513 (processing performed by determination means 222 for determining a motion level in a jaw movement, a tongue movement, or a throat movement of a subject based on a pattern contained in a processed myoelectric signal).

### DESCRIPTION OF EMBODIMENTS

The embodiments of the invention are explained hereinafter with reference to the drawings.

As used herein, “about” means that the subsequent number is within the range of the number±10%.

### 1. Myoelectric Device

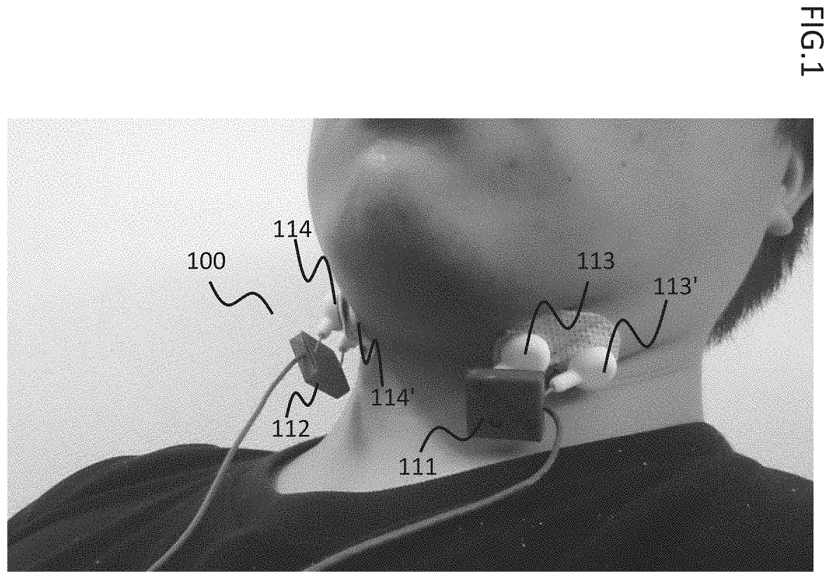

FIG. 1 shows a myoelectric device 100 fitted onto a subject.

The myoelectric device 100 comprises a first myoelectric sensor 111 and a second myoelectric sensor 112. The first myoelectric sensor 111 comprises a pair of measurement electrodes 113, 113′ and a reference electrode (not shown). The second myoelectric sensor 112 comprises a pair of measurement electrodes 114, 114′ and a reference electrode (not shown). The first myoelectric sensor 111 detects a myoelectric signal from an electric signal measured by the pair of measurement electrodes 113, 113′ and the reference electrode. The second myoelectric sensor 112 detects a myoelectric signal from an electric signal measured by the pair of measurement electrodes 114, 114′ and the reference electrode. A myoelectric signal is a signal indicating an activity of a muscle in the body.

The pair of measurement electrodes 113, 113′ and the pair of measurement electrodes 114, 114′ of the myoelectric device 100 are attached to a position on the body of a subject (e.g., position around the throat of the subject) where a myoelectric signal indicating activities of at least two muscles among a jaw muscle, a tongue muscle, and a throat muscle can be detected. For example, the pair of measurement electrodes 113, 113′ is attached to the left side of the Adam's apple, and the pair of measurement electrodes 114, 114′ is attached to the right side of the Adam's apple. The pair of measurement electrodes 113, 113′ and the pair of measurement electrodes 114, 114′ are preferably symmetrical to the left and right with respect to the Adam's apple. The position on the body of a subject where the pair of measurement electrodes 113, 113′ and the pair of measurement electrodes 114, 114′ are attached can be any position, as long as it is a position where a myoelectric signal indicating activities of at least two muscles among a jaw muscle, tongue muscle, and throat muscle can be detected, but is preferably a position where a myoelectric signal indicating activities of a jaw muscle, tongue muscle, and throat muscle can be detected. This is because three motions, i.e., motion of a jaw movement, motion of a tongue movement, and motion of a throat movement, would be identifiable by the processing discussed below. Such a position on the body of a subject can be, for example, the cheek, side of the neck, or the like as well as a position around the throat of the subject.

Reference electrodes of the first myoelectric sensor 111 and the second myoelectric sensor 112 can be the same reference electrode or separate reference electrodes. A reference electrode can be attached to, for example, the back of the neck of the subject.

The first myoelectric sensor 111 can detect a myoelectric signal indicating activities of at least two muscles among a muscle on the left side of the jaw, a muscle of the left side of the tongue, and a muscle of the left side of the throat by a reference electrode and the pair of measurement electrodes 113, 113′ attached to the left side of the throat of the subject. Preferably, the first myoelectric sensor 111 detects a myoelectric signal indicating activities of a muscle on the left side of the jaw, a muscle of the left side of the tongue, and a muscle of the left side of the throat. The second myoelectric sensor 112 can detect a myoelectric signal indicating activities of at least two muscles among a muscle on the right side of the jaw, a muscle of the right side of the tongue, and a muscle of the right side of the throat by a reference electrode and the pair of measurement electrodes 114, 114′ attached to the right side of the throat of the subject. Preferably, the second myoelectric sensor 112 detects a myoelectric signal indicating activities of a muscle on the right side of the jaw, a muscle of the right side of the tongue, and muscle of the right side of the throat.

Myoelectric signals indicating activities of a plurality of muscles coexist in myoelectric signals detected by the myoelectric device 100. Without any processing, it is impossible to identify the motion from which the myoelectric signal has originated. In this regard, a myoelectric signal detected by the myoelectric device 100 is processed by the system of the invention.

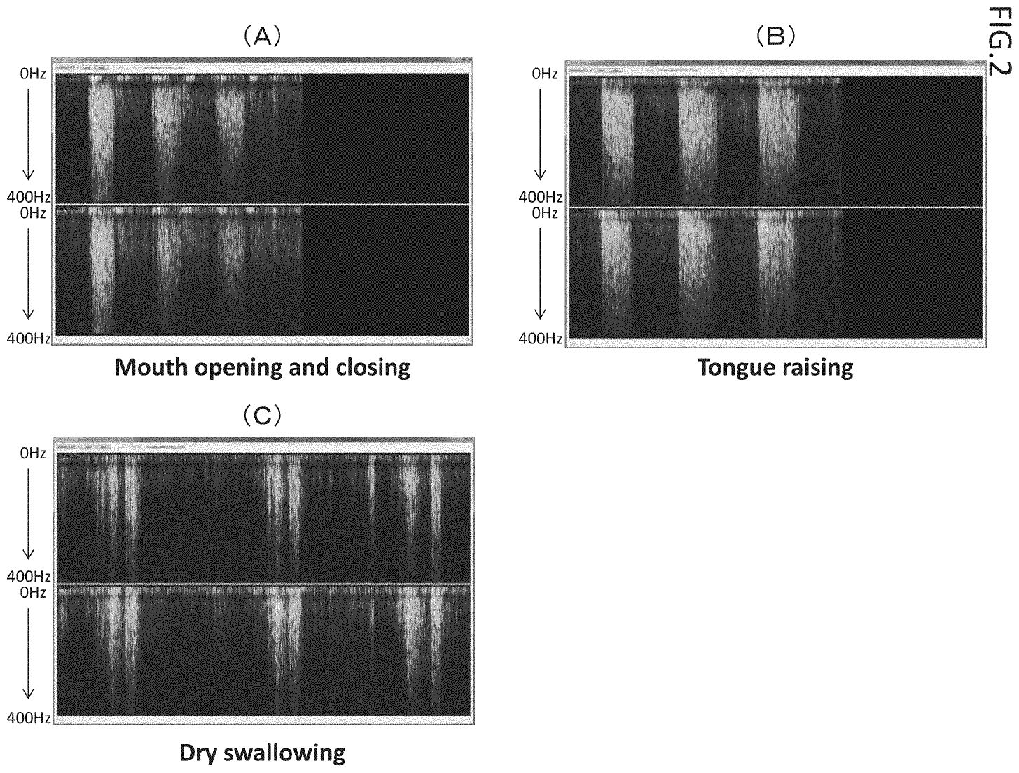

(A) in FIG. 2 is a graph showing an example of an output after the system of the invention has applied predetermined processing on a myoelectric signal detected by the myoelectric device 100 upon a motion of a jaw movement of a subject. (B) in FIG. 2 is a graph showing an example of an output after the system of the invention has applied predetermined processing on a myoelectric signal detected by the myoelectric device 100 upon a motion of a tongue movement of a subject. (C) in FIG. 2 is a graph showing an example of an output after the system of the invention has applied predetermined processing on a myoelectric signal detected by the myoelectric device 100 upon a motion of a throat movement of a subject.

Examples of the predetermined processing performed by the system of the invention include frequency analysis processing such as short-time FFT. A time series data for a frequency distribution can be obtained with such frequency analysis processing. (A) in FIG. 2 shows time series data for a frequency distribution obtained from myoelectric signals detected when a subject has opened and closed the mouth (mouth opening and closing motion) three times. (B) in FIG. 2 shows time series data for a frequency distribution obtained from myoelectric signals detected when a subject has pushed the tongue against the upper jaw (tongue raising motion) three times. (C) in FIG. 2 shows time series data for a frequency distribution obtained from myoelectric signals detected when a subject has swallowed without anything in the mouth (dry swallowing motion) three times. In (A) to (C) of FIG. 2, the horizontal axis represents the time, and the vertical axis represents frequency. The top of the vertical axis is 0 Hz, and the bottom of the vertical axis is 400 Hz. The color on the graph represents the intensity of frequency components. Darker color indicates weaker frequency component, and lighter color indicates stronger frequency component. In each of (A) to (C) in FIG. 2, the graph on the top side shows data from an output of the myoelectric sensor 111 attached to the left side of the throat of the subject, and the graph on the bottom side shows data from an output of the myoelectric sensor 112 attached to the right side of the throat of the subject.

The frequency distributions of myoelectric signals in (A) to (C) in FIG. 2 are compared. For the mouth opening and closing motion, frequencies of myoelectric signals are evenly distributed, especially strongly at about 50 to about 200 Hz, while widely distributed throughout the entire frequency band of myoelectric signals. For the tongue raising motion, frequencies of myoelectric signals are distributed strongly at about 50 to 200 Hz and weakly at high frequency bands above about 200 Hz, with about 100 Hz as the center. For the dry swallowing motion, frequencies of myoelectric signals are distributed especially strongly at about 100 Hz and weakly at high frequency bands above about 150 Hz.

The changes in time series of the frequency distributions of myoelectric signals in (A) to (C) in FIG. 2 are compared. For the mouth opening and closing motion, the frequency decreases over time. For the tongue raising motion, the frequency tends to not decrease over time. For the dry swallowing motion, there are two large peaks.

In this manner, an output after the system of the invention has applied predetermined processing on myoelectric signals detected by the myoelectric device 100 contains a specific pattern that can be matched to at least a motion of a jaw movement, a motion of a tongue movement, or a motion of a throat movement. The detected myoelectric signal-originating motion can be recognized as a motion of a jaw movement, a motion of a tongue movement, or a motion of a throat movement by recognizing these patterns.

Recognition of whether the detected myoelectric signal-originating motion is a motion of a jaw movement, a motion of a tongue movement, or a motion of a throat movement can be utilized in, for example, evaluation or rehabilitation of a motor function for swallowing or chewing.

For example, the system of the invention can determine the motion level in a jaw movement, tongue movement, or throat movement by learning the patterns of a motion of a jaw movement, a motion of a tongue movement, and a motion of a throat movement of a large number of healthy individuals as supervisory patterns and utilizing the result of matching the patterns of a motion of a jaw movement, a motion of a tongue movement, and a motion of a throat movement of a subject with the supervisory patterns. Determination of a motion level can quantitatively determine the size of a motion performed by a subject based on the size of motion of healthy individuals, or qualitatively determine whether the motion performed by the subject is normal. Medical practitioners such as physicians and rehab advisers can ultimately evaluate, judge, and determine whether the motor function for swallowing or chewing of a subject is normal by using an output from the system of the invention as an indicator.

For example, the system of the invention can quantify the degree of motor dysfunction or impairment of the jaw, tongue, or throat by utilizing the result of matching the patterns of a motion of a jaw movement, a motion of a tongue movement, and a motion of a throat movement of a subject with supervisory patterns, based on a detected myoelectric signal. Medical practitioners (physicians, rehab advisers, etc.) can provide appropriate guidance for rehabilitation that is in alignment with the site and degree of motor dysfunction or impairment by using an output from the system of the invention as an indicator. For example, a detected myoelectric signal can be utilized as an indicator to evaluate whether a patient can perform a motion properly by administering training to a patient for a specific motion among a jaw movement, tongue movement, and throat movement for rehabilitation. To show whether a subject is able to perform a motion properly, an avatar of the subject can be displayed on a monitor and make the avatar of the subject express the extent of motion based on the detected myoelectric signal. This enables the subjects to objectively see the actual extent of the motion intended by themselves.

For example, the precision of determining the motion level in a jaw movement, tongue movement, or throat movement can be improved by learning patterns of a motion of a jaw movement, a motion of a tongue movement, and a motion of a throat movement of a large number of healthy individuals as supervisory patterns. However, it should be noted that evaluation of a motor function for swallowing or chewing of a subject by medical practitioners discussed above is intended to assist the medical practitioners, so that the precision of determining a motion level can be somewhat low (e.g., 60%, 70%, or the like).

### 2. Configuration of a System for Recognizing Myoelectric Signal-Originating Motion

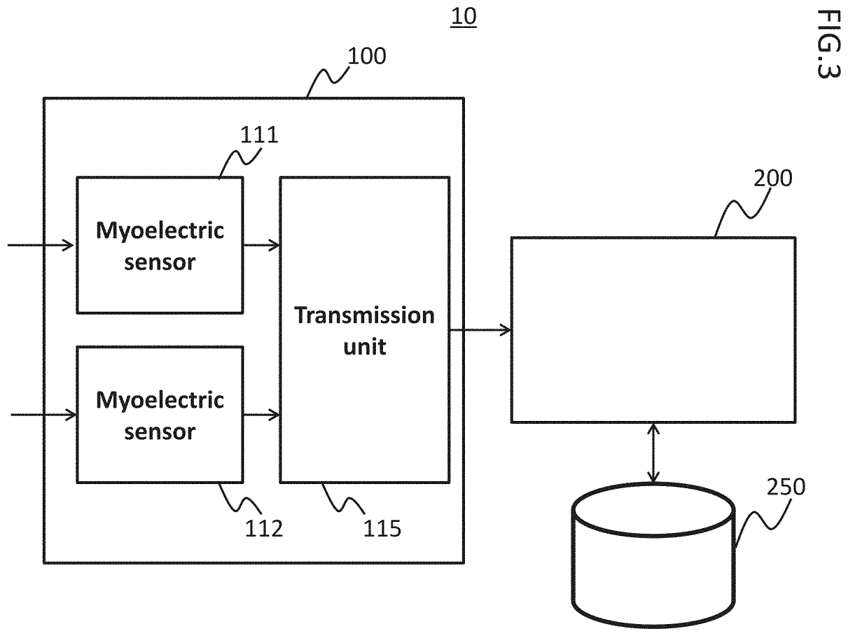

FIG. 3 depicts an example of a configuration of system 10 for recognizing a myoelectric signal-originating motion. In this regard, myoelectric signal-originating motions comprise at least two of a motion of a jaw movement, a motion of a tongue movement, and a motion of a throat movement, but preferably comprises three motions, i.e., a motion of a jaw movement, a motion of a tongue movement, and a motion of a throat movement.

The system 10 comprises a myoelectric device 100 and a computer apparatus 200. A database unit 250 is connected to the computer apparatus 200. The myoelectric device 100 and the computer apparatus 200 are connected via a network. In this regard, the network can be any type of network. For example, the myoelectric device 100 and the computer apparatus 200 can communicate with each other via the Internet or via LAN.

The myoelectric device 100 comprises two myoelectric sensors 111, 112 and a transmission unit 115.

The myoelectric sensor 111 comprises a pair of measurement electrodes 113, 113′ and a reference electrode, as shown in FIG. 1. The myoelectric sensor 112 comprises a pair of measurement electrodes 114, 114′ and a reference electrode, as shown in FIG. 1. The myoelectric sensors 111, 112 can be any detection means that is capable of detecting a myoelectric signal indicating the activity of a muscle in the body. For example, the myoelectric sensors 111, 112 can comprise a primary amplifier, a high pass filter, a low pass filter, a notch filter, and a secondary amplifier for the detection of myoelectric signals. Primary and secondary amplifiers are used for amplifying a signal. A high pass filter is used for attenuating a signal with a frequency lower than a predetermined frequency, e.g., a signal with a frequency lower than about 10 Hz. A low pass filter is used for attenuating a signal with a frequency higher than a predetermined frequency, e.g., a signal with a frequency higher than about 400 Hz. A notch filter is used to attenuate a signal with a frequency in a predetermined range, e.g., 50 to 60 Hz AC noise, which is a typical electrical noise. A band elimination filter can also be used in place of a notch filter.

The transmission unit 115 is configured to transmit a signal out of the myoelectric device 100. The transmission unit 115 transmits a signal out of the myoelectric device 100 by wireless or wired transmission. For example, the transmission unit 115 can transmit a signal by utilizing a wireless LAN such as Wi-Fi. For example, the transmission unit 115 can transmit a signal by utilizing a short range wireless communication such as Bluetooth®. The transmission unit 115 transmits a myoelectric signal indicating activities of a plurality of muscles in the body including a jaw muscle, tongue muscle, and throat muscle detected by a myoelectric sensor to the computer apparatus 200.

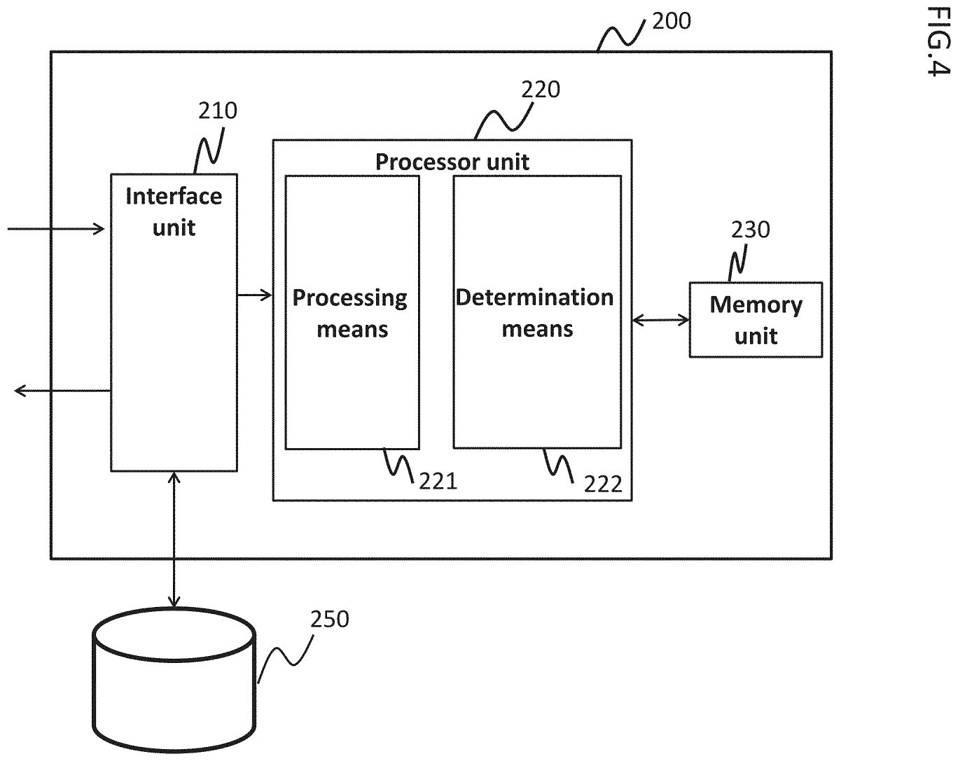

FIG. 4 depicts an example of a configuration of the computer apparatus 200.

The computer apparatus 200 comprises an interface unit 210, a processor unit 220, and a memory unit 230. As discussed above, the database unit 250 is connected to the computer apparatus 200.

The interface unit 210 is configured to control communication of the computer apparatus 200 with the outside. The interface unit 210 can transmit information out of the computer apparatus 200 and receive information from the outside of the computer apparatus 200. For example, the interface unit 210 controls communication with the myoelectric device 100 or the database unit 250. The interface unit 210 can control communication by any method. Communication can be wired communication or wireless communication.

The processor unit 220 controls the operation of the entire computer apparatus 200. The processor unit 220 reads out a program stored in the memory unit 230 and executes the program. This allows the computer apparatus 200 to function as an apparatus that executes desired steps.

The processor unit 220 comprises processing means 221 and determination means 222.

The processing means 221 is configured to output a processed myoelectric signal by applying predetermined processing on an input myoelectric signal. Predetermined processing comprises, for example, signal waveform drawing processing, signal intensity extraction processing, and frequency analysis processing. Signal waveform drawing processing is processing for drawing a waveform of a detected myoelectric signal in time series. The waveform itself of a myoelectric signal can be studied from such an output. Signal intensity extraction processing is processing for extracting the intensity of a detected myoelectric signal to obtain time series data. The intensity of a myoelectric signal is a value that is proportional to the force exerted by a myoelectric signal-originating muscle. Signal intensity extraction processing can be, for example, a combination of full-wave rectification processing and moving average processing. Frequency analysis processing is processing for obtaining time series data for a frequency distribution of a detected myoelectric signal. Since the frequency distribution of myoelectric signals varies by muscle, differences in the primary active muscle can be observed by performing frequency analysis processing. With frequency analysis processing, muscle activities can also be observed even if the myoelectric signal intensity is minute. Frequency analysis processing comprises, for example, short-time FFT. Short-time FFT applies FFT on a myoelectric signal obtained in any short period of time (e.g., about 0.01 seconds to about 1 second, preferably about 0.1 seconds or about 128/1600 seconds). Shorter time interval leads to higher response rate (rate of an output responding to an input), while leading to lower frequency resolution, so that stability of a signal is not guaranteed. In contrast, longer time interval (e.g., about 256/1600 seconds or longer) leads to higher frequency resolution, but a lower response rate. Thus, the precision in time decreases, so that evaluation in real time would not be possible. Longer time interval also increases the amount of calculation, resulting in higher hardware cost. For example, if the time interval is about 0.1 seconds, the response rate and frequency resolution can be maintained to the extent that a pattern contained in a myoelectric signal can identify a pattern of a motion of a jaw movement, a pattern of a motion of a tongue movement, or a pattern of a motion of a throat movement, so that signal stability and real-time evaluation can both be attained with relatively low amount of calculation. For example, FFT is performed at an interval of about 0.1 seconds, i.e., FFT is applied on a myoelectric signal obtained at 0 to about 0.1 seconds, FFT is applied on a myoelectric signal obtained at about 0.1 to about 0.2 seconds, FFT is applied on a myoelectric signal obtained at about 0.2 to about 0.3 seconds . . . . FFT is applied on a myoelectric signal obtained at (n−0.1) to n seconds. Time series data for a frequency distribution can be obtained by connecting a plurality of frequency distributions obtained by short-time FFT.

The determination means 222 is configured to determine which one or more of a plurality of patterns of motions comprising at least two of a pattern of a motion of a jaw movement, a pattern of a motion of a tongue movement, and a motion of a throat movement is a pattern contained in the processed myoelectric signal outputted by the processing means 221. The determination means 222 determines which one or more of a plurality of patterns of motions comprising at least two of a pattern of a motion of a jaw movement, a pattern of a motion of a tongue movement, and pattern of a motion of a throat movement is a pattern contained in the processed myoelectric signal by, for example, matching a pattern with a supervisory pattern of a motion stored in the database unit 250. In this regard, the determination means 222 can be configured to comprise learning means for learning at least two of a supervisory pattern of a motion of a jaw movement, a supervisory pattern of a motion of a tongue movement, and a supervisory pattern of a motion of a throat movement, calculation means for calculating a degree of similarity between a processed myoelectric signal and the supervisory pattern, and judgment means for judging whether the degree of similarity exceeds a predetermined threshold value. The learning means can also learn a supervisory pattern for no motion being performed in addition to a supervisory pattern of a motion of a jaw movement, a supervisory pattern of a motion of a tongue movement, and a supervisory pattern of a motion of a throat movement. This is because even if a subject is not performing a motion, a myoelectric signal is not necessarily zero due to a force that is unconsciously exerted, effect of environmental noise, or the like. This enables the determination means 222 to determine that a pattern contained in a processed myoelectric signal is a pattern for no motion being performed.

The determination means 222 can determine which one of the plurality of patterns of motions is each of the plurality of patterns contained in a processed myoelectric signal, even when the processed myoelectric signal contains a plurality of patterns. This enables recognition of such motions even if a myoelectric signal originates from a composite motion.

The determination means 222 can be configured to determine a motion level or evaluate the presence or absence of an impairment in a jaw movement, tongue movement, or throat movement of a subject, based on a pattern contained in a processed myoelectric signal outputted from the processing means 221, in addition to, or instead of, the aforementioned configuration. For example, the determination means 222 determines a motion level or evaluates the presence or absence of an impairment in a jaw movement, tongue movement, or throat movement of a subject, based on a pattern contained in a processed myoelectric signal by matching a pattern with a supervisory pattern of motion stored in the database unit 250. In this regard, the determination means 222 can be configured to comprise learning means for learning a supervisory pattern of a motion of a jaw movement, a supervisory pattern of a motion of a tongue movement, or a supervisory pattern of a motion of a throat movement, and calculation means for calculating a degree of similarity between a processed myoelectric signal and the supervisory pattern. The determination means 222 can be configured to further comprise judgment means for judging whether the degree of similarity exceeds a predetermined threshold value. For example, the determination means 222 can determine that there is an impairment in a jaw movement, tongue movement, or throat movement of a subject if the degree of similarity does not exceed a predetermined threshold value. The system 10 comprising such determination means 222 can be used to evaluate a movement of a subject.

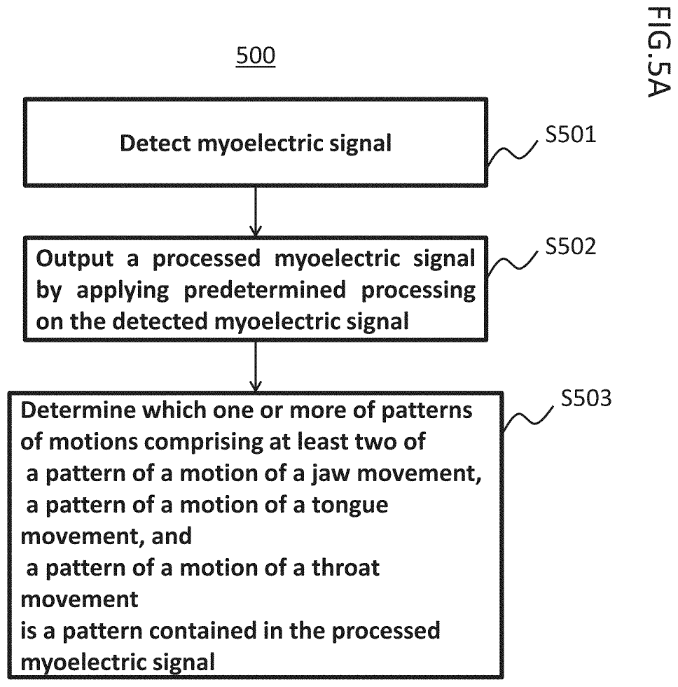

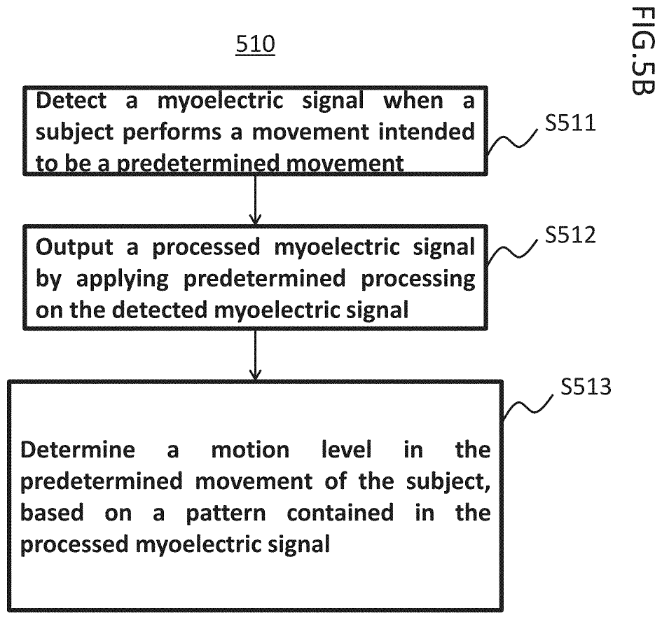

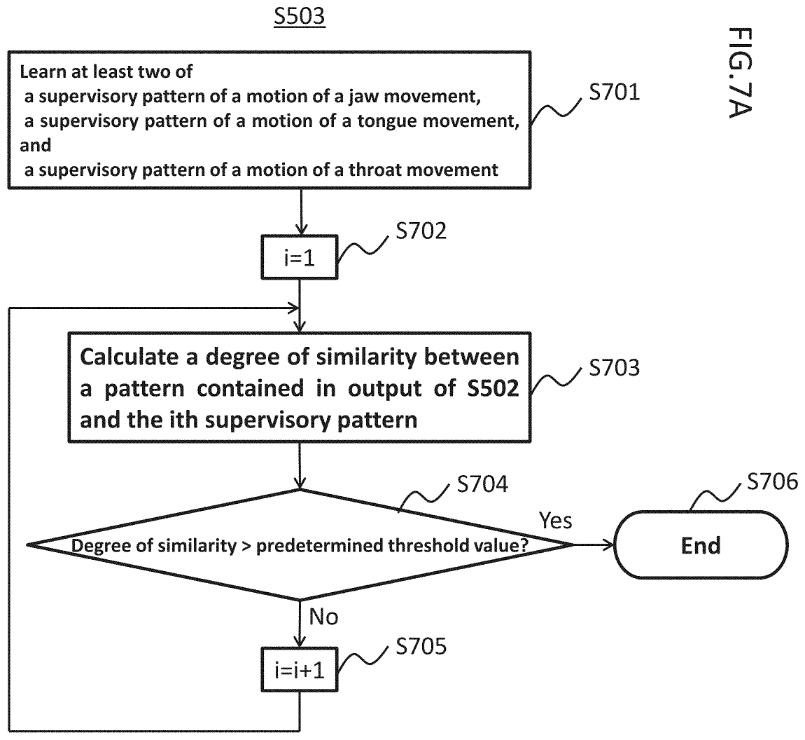

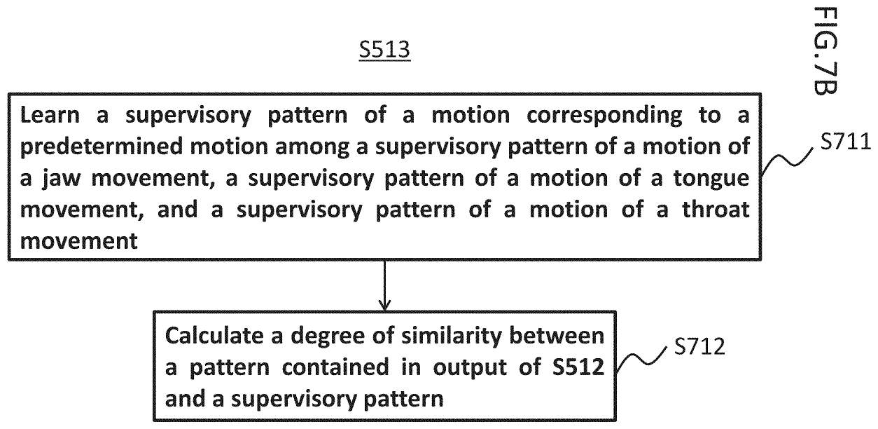

The memory unit 230 stores a program required to execute processing, data required to execute the program, and the like. For example, a program for materializing processing for recognizing a myoelectric signal-originating motion (e.g., processing discussed below in FIGS. 5A and 7A), processing for evaluating a movement of a subject (e.g., processing discussed below in FIGS. 5B and 7B), or processing for constructing a classifier used for evaluating a movement of a subject can be stored in the memory unit 230. In this regard, the program can be stored in the memory unit 230 by any manner. For example, a program can be preinstalled in the memory unit 230. Alternatively, a program can be installed in the memory unit 230 by downloading the program through a network, or installed in the memory unit 230 via a storage medium such as an optical disk or USB.

For example, a pattern of a motion of a jaw movement, a pattern of a motion of a tongue movement, a pattern of a motion of a throat movement, or a pattern for no motion being performed recognized from a myoelectric signal transmitted from the myoelectric device 100 can be stored in the database unit 250 while being associated with a user of the myoelectric device 100. A supervisory pattern of a motion of a jaw movement, a supervisory pattern of a motion of a tongue movement, a supervisory pattern of a motion of a throat movement, or a supervisory pattern for no motion being performed learned by learning means of the determination means 222 can also be stored in the database unit 250. The database unit 250 can be configured to store a weighting coefficient for each node of a neural network discussed below.

The example depicted in FIG. 3 has explained an example of the myoelectric device 100 comprising two myoelectric sensors 111, 112, but the number of myoelectric sensors is not limited thereto. The myoelectric device 100 comprising three or more myoelectric sensors is also within the scope of the present invention.

While each constituent element of the computer apparatus 200 is provided within the computer apparatus 200 in the examples depicted in FIGS. 3 and 4, the present invention is not limited thereto. Any of the constituent elements of the computer apparatus 200 can be provided outside of the computer apparatus 200. For example, if each of the processor unit 220 and the memory unit 230 is comprised of separate hardware parts, each hardware part can be connected via any network. In this regard, the network can be any type of network. For example, each hardware part can be connected via LAN, wireless connection, or wired connection.

In the examples depicted in FIGS. 3 and 4, the database unit 250 is provided external to the computer apparatus 200, but the present invention is not limited thereto. The database unit 250 can be provided inside the computer apparatus 200. In this regard, the database unit 250 can be implemented by the same storing means as the storing means implementing the memory unit 230, or by storage means that is different from the storing means implementing the memory unit 230. In either case, the database unit 250 is configured as a storing unit for the computer apparatus 200. The configuration of the database unit 250 is not limited to a specific hardware configuration. For example, the database unit 250 can be configured with a single hardware part or a plurality of hardware parts. For example, the database unit 250 can be configured as an external hard disk apparatus of the computer apparatus 200 or as a storage on the cloud connected via a network.

### 3. Processing for Recognizing Myoelectric Signal-Originating Motion

FIG. 5A depicts an example of processing 500 for recognizing a myoelectric signal-originating motion. This processing is executed by the system 10. In this regard, a myoelectric signal-originating motion comprises at least two of a motion of a jaw movement, a motion of a tongue movement, and a motion of a throat movement, but preferably comprises three motions, i.e., a motion of a jaw movement, a motion of a tongue movement, and a motion of a throat movement.

In step S501, the first myoelectric sensor 111 and the second myoelectric sensor 112 of the myoelectric device 100 detect a myoelectric signal indicating activities of a plurality of muscles. The first myoelectric sensor 111 detects a myoelectric signal using a pair of measurement electrodes 113, 113′ and a reference electrode. The second myoelectric sensor 112 detects a myoelectric signal using a pair of measurement electrodes 114, 114′ and a reference electrode. If such myoelectric signals are detected, the myoelectric device 100 transmits the detected myoelectric signals to the computer apparatus 200 via the transmission unit 115. The computer apparatus 200 receives the detected myoelectric signals via the interface unit 210.

In step S502, the processing means 221 of the processor unit 220 of the computer apparatus 200 applies predetermined processing on a myoelectric signal detected by the myoelectric device 100 to output a processed myoelectric signal. The predetermined processing comprises, for example, signal waveform drawing processing, signal intensity extraction processing, and frequency analysis processing. The processed myoelectric signal outputted from the processing means 221 is provided to the determination means 222.

In step S503, the determination means 222 of the processor unit 220 determines which one or more of patterns of motions comprising at least two of a pattern of a motion of a jaw movement, a pattern of a motion of a tongue movement, and a pattern of a motion of a throat movement is a pattern contained in the processed myoelectric signal. A processed myoelectric signal comprises a specific pattern that can be matched at least to a motion of a jaw movement, a motion of a tongue movement, or a motion of a throat movement. The determination means 222 determines which one or more of patterns of motions comprising at least two of a pattern of a motion of a jaw movement, a pattern of a motion of a tongue movement, and a pattern of a motion of a throat movement is this pattern. For example, the determination means 222 determines which one or more of patterns of motions comprising at least two of a pattern of a motion of a jaw movement, a pattern of a motion of a tongue movement, and a pattern of a motion of a throat movement is a pattern contained in the processed myoelectric signal by the processing discussed below in FIG. 7A, In this regard, the determination means 222 can also determine that the pattern contained in the processed myoelectric signal is a pattern for no motion being performed. Alternatively, the determination means 222 can also determine that the patterns contained in the processed myoelectric signal are patterns for a plurality of motions, and determine which pattern corresponds to each of the patterns of the plurality of motions.

It is possible to recognize, with the processing discussed above, whether a myoelectric signal-originating motion is a motion of a jaw movement, a motion of a tongue movement, a motion of a throat movement, or a composite motion.

Since it is possible to recognize whether a myoelectric signal-originating motion is a motion of a jaw movement, a motion of a tongue movement, a motion of a throat movement, or a composite motion, the system 10 of the invention can, for example, evaluate whether there is an impairment in a movement of a subject or not, or evaluate a motion level of a movement of a subject based thereon.

FIG. 5B depicts an example of processing 510 for evaluating a movement of a subject. This processing is executed in the system 10.

In step S511, the first myoelectric sensor 111 and the second myoelectric sensor 112 of the myoelectric device 100 detect a myoelectric signal indicating an activity of a muscle when a subject performs a movement intending a predetermined movement comprising a jaw movement, a tongue movement, or a throat movement. Since this step is the same as step S501 of processing 500, the explanation is omitted.

In step S512, the processing means 221 of the processor unit 220 of the computer apparatus 200 outputs a processed myoelectric signal by applying predetermined processing on a myoelectric signal detected by the myoelectric device 100. Since this step is the same as step S502 of processing 500, the explanation is omitted.

In step S513, the determination means 222 of the processor unit 220 determines a motion level in a predetermined movement of a subject based on a pattern contained in a processed myoelectric signal. The processed myoelectric signal at least comprises a specific pattern that can be matched to a motion of a jaw movement of a healthy individual, a motion of a tongue movement of a healthy individual, or a motion of a throat movement of a healthy individual. The determination means 222 determines a motion level in a predetermined movement comprising a jaw movement, a tongue movement, or a throat movement of a subject based on such patterns. The determination means 222, for example, calculates the degree of similarity between a pattern contained in a processed myoelectric signal and a pattern of a motion of a jaw movement of a healthy individual, a pattern of a motion of a tongue movement of a healthy individual, or pattern of a motion of a throat movement of a healthy individual by the processing discussed below in FIG. 7B and determines a motion level in a predetermined movement comprising a jaw movement, tongue movement, or throat movement of a subject based on the degree of similarity. For example, the determination means 222 can be configured to evaluate the presence or absence of an impairment in a predetermined movement comprising a jaw movement, tongue movement, or throat movement of a subject based on the degree of similarity.

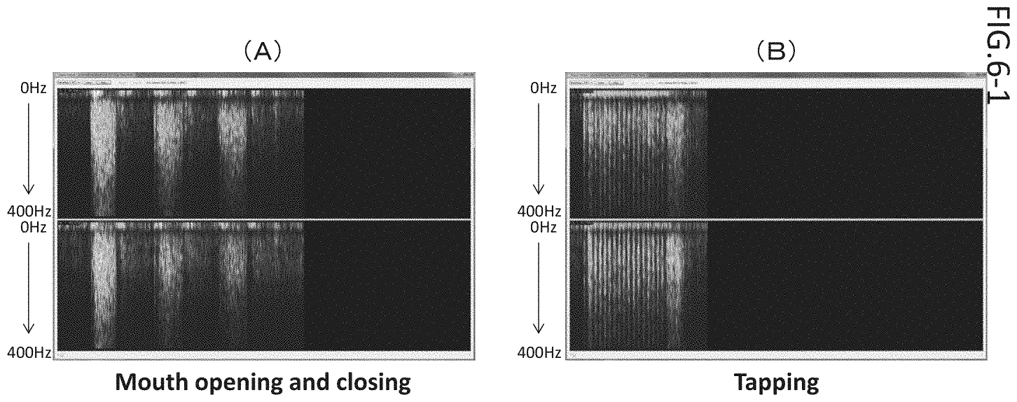

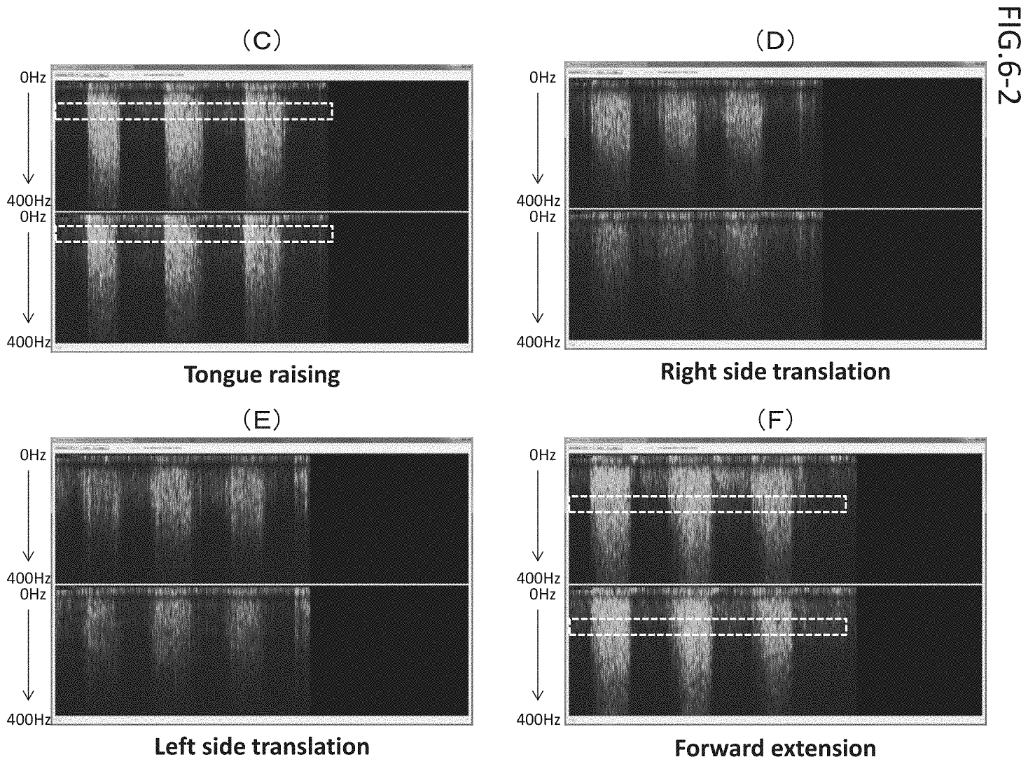

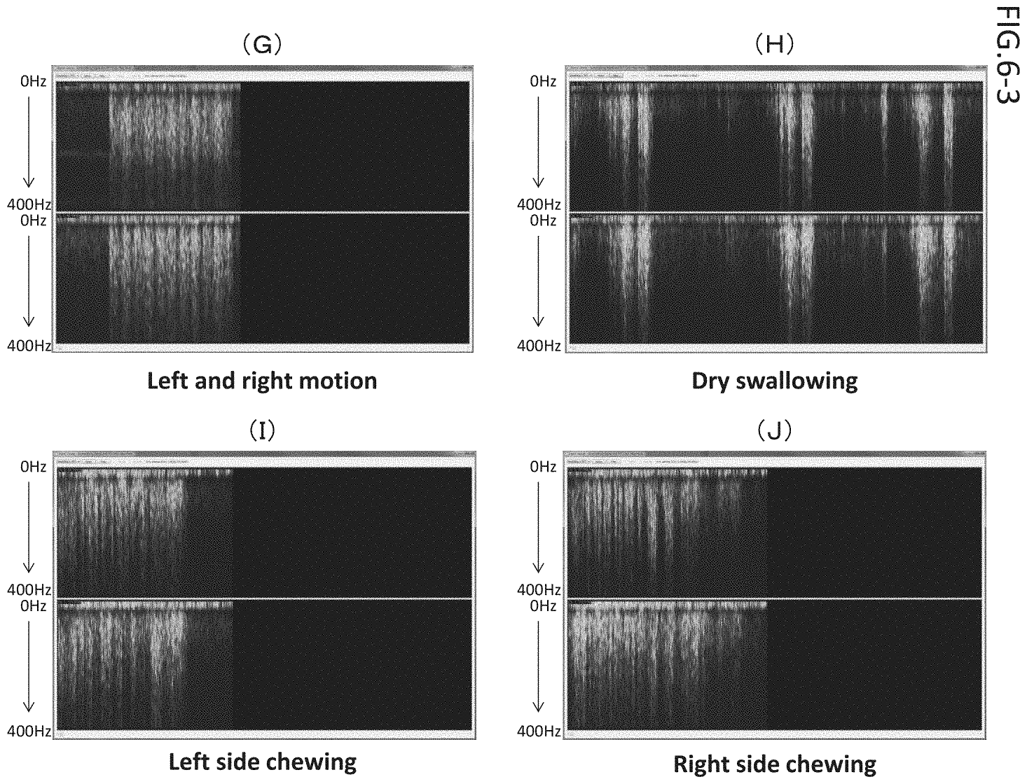

(A) to (J) in FIG. 6 are graphs showing an example of an output after frequency analysis processing by processing means 221 in step S502. In (A) to (J) in FIG. 6, the horizontal axis represents time, and the vertical axis represents frequency in the same manner as (A) to (C) in FIG. 2. The top of the vertical axis is 0 Hz, and the bottom of the vertical axis is about 400 Hz. The color on the graphs represents the intensity of frequency components. Darker color indicates weaker frequency component, and lighter color indicates stronger frequency component. In each of (A) to (J) in FIG. 6, the graph on the top side shows data from an output of the myoelectric sensor 111 attached to the left side of the throat of the subject, and the graph on the bottom side shows data from an output of the myoelectric sensor 112 attached to the right side of the throat of the subject.

(A) in FIG. 6 shows time series data for a frequency distribution obtained from myoelectric signals detected when a subject has opened and closed the mouth (mouth opening and closing motion) three times. For the mouth opening and closing motion, frequencies of myoelectric signals are evenly distributed, especially strongly at about 50 to about 200 Hz, while widely distributed throughout the entire frequency band of myoelectric signals of about 10 to about 400 Hz.

(B) in FIG. 6 shows time series data for a frequency distribution obtained from myoelectric signals detected when a subject has consecutively struck the top and bottom teeth against each other (tapping motion). For the tapping motion, frequencies of myoelectric signals are evenly distributed, especially strongly at about 50 to about 200 Hz, while widely distributed throughout the entire frequency band of myoelectric signals of about 10 to about 400 Hz. For a tapping motion, the frequency distribution and signal intensity vary at the same cycle as tapping (e.g., 0.1 seconds, 0.2 seconds, 0.3 seconds, 0.5 seconds, etc.)

(C) in FIG. 6 shows time series data for a frequency distribution obtained from myoelectric signals detected when a subject has pushed the tongue against the upper jaw (tongue raising motion) three times. For the tongue raising motion, frequencies of myoelectric signals are distributed strongly at about 50 to 200 Hz and weakly at high frequency bands above about 200 Hz, with about 100 Hz as the center.

(D) in FIG. 6 shows time series data for a frequency distribution obtained from myoelectric signals detected when a subject has translated the tongue to the right side (right side translation motion) three times. For the right side translation motion, frequencies of myoelectric signals are distributed strongly at about 50 to 200 Hz and weakly at high frequency bands above about 200 Hz, with about 100 Hz as the center.

(E) in FIG. 6 shows time series data for a frequency distribution obtained from myoelectric signals detected when a subject has translated the tongue to the left side (left side translation motion) three times. For the left side translation motion, frequencies of myoelectric signals are distributed strongly at about 50 to 200 Hz and weakly at high frequency bands above about 200 Hz, with about 100 Hz as the center.

(F) in FIG. 6 shows time series data for a frequency distribution obtained from myoelectric signals detected when a subject has pushed the tongue forward (forward extension motion) three times. For the forward extension motion, frequencies of myoelectric signals are distributed to be high around about 150 Hz.

(G) in FIG. 6 shows time series data for a frequency distribution obtained from myoelectric signals detected when a subject has consecutively translated the tongue to the left and right (left and right movement motion) three times. For the left and right motion, frequencies of myoelectric signals are distributed strongly at about 50 to 200 Hz and weakly at high frequency bands above about 200 Hz, with about 100 Hz as the center. For the left and right motion, the frequency distribution and signal intensity vary at the same cycle as the left and right motion (e.g., about 0.5 seconds, about 1 second, etc.)

(H) in FIG. 6 shows time series data for a frequency distribution obtained from myoelectric signals detected when a subject has swallowed without anything in the mouth (dry swallowing motion) three times. For the dry swallowing motion, frequencies of myoelectric signals are distributed especially strongly at about 100 Hz and weakly at high frequency bands above about 150 Hz. For the dry swallowing motion, the frequency and signal intensity vary so that there are two consecutive peaks in the time series.

(I) in FIG. 6 shows time series data for a frequency distribution obtained from myoelectric signals detected when a subject has chewed a chew managing food on the left side (left side chewing motion). For the left side chewing motion, frequencies of myoelectric signals are distributed weakly at high frequency bands above about 200 Hz, with about 100 Hz as the center. For the left side chewing motion, the frequency distribution and signal intensity vary at the same cycle as the chewing (e.g., about 0.1 seconds, about 0.2 seconds, about 0.3 seconds, about 0.5 seconds, etc.)

(J) in FIG. 6 shows time series data for a frequency distribution obtained from myoelectric signals detected when a subject has chewed a chew managing food on the right side (right side chewing motion). For the right side chewing motion, frequencies of myoelectric signals are distributed weakly at high frequency bands above about 200 Hz, with about 100 Hz as the center. For the right side chewing motion, the frequency distribution and signal intensity vary at the same cycle as the chewing (e.g., about 0.1 seconds, about 0.2 seconds, about 0.3 seconds, about 0.5 seconds, etc.)

(A) in FIG. 6 shows the same data as (A) in FIG. 2, (C) in FIG. 6 shows the same data as (B) in FIG. 2, and (H) in FIG. 6 shows the same data as (C) in FIG. 2.

As discussed above in reference to (A) to (C) in FIG. 2, a mouth opening and closing motion, a tongue raising motion, and a dry swallowing motion can be distinguished, for example, by the frequency distribution of myoelectric signals and the change in time series thereof.

For example, it can be understood by comparing the intensities of frequency components in (C) to (E) in FIG. 6 that the intensities of myoelectric signals in tongue raising motions and forward extension motions are higher than intensities of myoelectric signals in right side translation motions and left side translation motions. In this manner, a tongue raising motion and a forward extension motion can be distinguished from a right side translation motion and a left side translation motion by comparing the intensities of frequency components.

For example, it can be understood by comparing the frequency distributions in (C) and (E) in FIG. 6 that the band with a strong frequency component of a myoelectric signal in a tongue raising motion is in a low frequency band in the vicinity of 100 Hz (band surrounded by a dotted line in (C) in FIG. 6), while the band with a strong frequency component of a myoelectric signal in a forward extension motion is in a frequency band greater than 100 Hz (band surrounded by a dotted line in (F) in FIG. 6). In this manner, a tongue raising motion and a forward extension motion can be distinguished by comparing frequency distributions

For example, in view of comparing the changes in time series of the frequency distributions in (B), (I), and (in in FIG. 6, a tapping motion is the same as a left side chewing motion and a right side chewing motion in that a striped pattern of short intervals due to a jaw movement can be observed. However, since the tongue or throat muscle is also active in addition to the jaw muscle in a chewing motion, there are myoelectric signals due to a tongue or throat movement between the stripe patterns.

Table 1 summarizes examples of a characteristic of a signal intensity distribution, a characteristic of a frequency distribution, and a characteristic of time series of a pattern of a motion of a jaw movement, a pattern of a motion of a tongue movement, and a pattern of a motion of a throat movement obtained from time series data for frequency distributions shown in (A) to (H) of FIG. 6 and corresponding signal intensity distributions.

TABLE 1

| | | Characteristic | Characteristic | |
|---|---|---|---|---|
| | | of intensity | of frequency | Characteristic |
| Movement | Motion | distribution | distribution | of time series |
| Jaw | Mouth | Signal | Evenly | Decease over |
| movement | opening | intensity is | distributed, | time |
| | and | stronger | especially | |
| | closing | relative to | strongly near | |
| | | tongue | 50 to 200 Hz, | |
| | | movement | while widely | |
| | | | distributed | |
| | | | throughout the | |
| | | | entire | |
| | | | myoelectric | |
| | | | frequency band | |
| | Tapping | Same as above | Same as above | Change in |
| | | | | much shorter |
| | | | | intervals |
| | | | | relative to |
| | | | | mouth opening |
| | | | | and closing |
| Tongue | Tongue | Signal | Distributed | Tend not to |
| movement | raising | intensity is | strongly near | decrease over |
| | | weaker relative | 50 to 200 Hz | time |
| | | to jaw | and weakly at | |
| | | movement | high frequency | |
| | | | bands above | |
| | | | 200 Hz, with | |
| | | | around 100 Hz | |
| | | | as the center | |
| | Left side | Signal | Same as above | Same as above |
| | translation | intensity is | | |
| | | weaker overall | | |
| | | relative to | | |
| | | tongue raising; | | |
| | | left-right | | |
| | | balance is | | |
| | | right > left | | |
| | Right side | Signal | Same as above | Same as above |
| | translation | intensity is | | |
| | | weaker overall | | |
| | | relative to | | |
| | | tongue raising; | | |
| | | left-right | | |
| | | balance is | | |
| | | left > right | | |
| | Left and | Alternating | Same as left | Same as left |
| | right | movement to | | |
| | movement | left and right | | |
| | Forward | Have | Distributed | Have |
| | extension | characteristics | strongly in | characteristics |
| | | of both tongue | frequency band | of both tongue |
| | | raising and | higher than | raising and |
| | | mouth opening | tongue raising | mouth opening |
| | | and closing | | and closing |
| Throat | Dry | Signal | Especially | Have two large |
| movement | swal- | intensity is | strong near | peaks, and |
| | lowing | greatest | 100 Hz, but | space between |
| | | relative to | dramatically | two peaks are |
| | | other | weaker above | almost the |
| | | movements | 150 Hz | same |

As can be understood from Table 1, the signal intensity of a throat movement is the strongest, the signal intensity of a jaw movement is the second strongest, and the signal intensity of a tongue movement is the weakest. This is due to the following reasons. Since two pairs of measurement electrodes (113, 113′) (114, 114′) of the myoelectric sensors 11, 12 are attached around the throat, the myoelectric sensors 11, 12 more strongly detect myoelectric signals indicating activities of a throat muscle, and a throat muscle is the largest muscle among jaw muscle, tongue muscle, and throat muscle. In other words, the pattern of signal intensities can vary depending on the measurement position of the two pairs of measurement electrodes (113, 113′) (114, 114′).

For example, if the signal intensity distribution obtained from myoelectric signals detected from a subject chewing a chew managing food 5 times on one side (5 times chewing motion) is compared with the signal intensity distribution obtained from myoelectric signals detected from a subject chewing a chew managing food freely for 10 seconds (10 second chewing motion), the signal intensity from a 5 time chewing motion is stronger than the signal intensity from a 10 second chewing motion. This is because reduction of the number of chewing leads to forced swallowing, which puts a burden on a throat muscle.

The motion patterns discussed above are patterns observed mainly in healthy adults. Different motion patterns are exhibited in healthy children in some cases. For example, two consecutive peaks were not observed in the time series observed in healthy adults, but instead only one peak was observed in some cases in myoelectric signals obtained from children with respect to the change in time series of frequencies in a dry swallowing motion. This is understood to be because muscles of children are undeveloped so that only the muscle of one of the tongue and throat is used for the swallowing motion. It can be determined whether an adult pattern is observed or a child pattern is observed based on, for example, secondary sexual characteristics. For example, an adult pattern is observed in subjects who have developed secondary sexual characteristics, and a child pattern is observed in subjects who have yet to develop secondary sexual characteristics.

For example, the system 10 can recognize whether a myoelectric signal-originating motion of a subject is a motion of a jaw movement, a motion of a tongue movement, a motion of a throat movement, or a composite motion regardless of whether the subject is an adult or a child by switching between an operational mode for identifying an adult pattern and an operational mode for identifying a child pattern. Such an output result from the system 10 can be utilized in evaluation or rehabilitation of a motor function for swallowing/chewing in both adults and children.

FIG. 7A depicts an example of processing in step S503 (processing performed by determination means 222 for determining which one or more of patterns of motions comprising at least two of a pattern of a motion of a jaw movement, a pattern of a motion of a tongue movement, and a pattern of a motion of a throat movement correspond to a pattern contained in a processed myoelectric signal).

In step S701, learning means of the determination means 222 learns at least two of a supervisory pattern of a motion of a jaw movement, a supervisory pattern of a motion of a tongue movement, and a supervisory pattern of a motion of a throat movement. The determination means 222 learns, for example, characteristics of at least two among a supervisory pattern of a motion of a jaw movement, a supervisory pattern of a motion of a tongue movement, and a supervisory pattern of a motion of a throat movement received via the interface unit 210, and stores the characteristics in the database unit 250. A supervisory pattern is a pattern contained in a processed myoelectric signal obtained by applying processing similar to the processing applied by the processing means 221 discussed above on a myoelectric signal obtained from a healthy individual performing a motion of a jaw movement, a motion of a tongue movement, or a motion of a throat movement. A characteristic of a supervisory pattern of a motion of a jaw movement, a supervisory pattern of a motion of a tongue movement, and a supervisory pattern of a motion of a throat movement can comprise, for example, a characteristic of a signal intensity distribution, a characteristic of a frequency distribution, and a characteristic of time series as explained in Table 1. It can be identified whether a muscle closest to the position where a measurement electrode is fitted is active from the characteristic of a signal intensity distribution. The difference in the primary active muscle can be identified from a characteristic of a frequency distribution. The temporal change in frequency and intensity of a myoelectric signal can be identified from a characteristic of time series. A pattern of a motion of a jaw movement, a pattern of a motion of a tongue movement, and a pattern of a motion of a throat movement can be clearly distinguished by combining these characteristics. A characteristic of a supervisory pattern of a motion of a jaw movement, a supervisory pattern of a motion of a tongue movement, and a supervisory pattern of a motion of a throat movement can also comprise, for example, a characteristic of a signal waveform. In this regard, learning means can also learn a supervisory pattern for no motion being performed in addition to the supervisory patterns discussed above.

In step S702, variable i is defined as i=1.

In step S703, calculation means of the determination means 222 calculates the degree of similarity between a pattern contained in an output of the processing means 221 after processing in step S502 (processed myoelectric signal) and the ith supervisory pattern. In this regard, the ith supervisory pattern is one of the supervisory patterns learned in step S701. For example, the first supervisory pattern can be a supervisory pattern of a mouth opening and closing motion of a jaw movement, the second supervisory pattern can be a supervisory pattern of a tapping motion of a jaw movement, the third supervisory pattern can be a supervisory pattern of a tongue raising motion of a tongue movement, the fourth supervisory pattern can be a supervisory pattern of a right side translation motion of a tongue movement, the fifth supervisory pattern can be a supervisory pattern of a left side translation motion of a tongue movement, the sixth supervisory pattern can be a supervisory pattern of a forward extension motion of a tongue movement, the seventh supervisory pattern can be a supervisory pattern of a left and right motion of a tongue movement, and the eight supervisory pattern can be a supervisory pattern of a dry swallowing motion of a throat movement. The calculation means can calculate the degree of similarity between an output of the processing means 221 (processed myoelectric signal) and ith supervisory pattern using a known similarity calculating technique such as normalized cross-correlation.

In step S704, it is judged whether the degree of similarity calculated in step S703 exceeds a predetermined threshold value. The predetermined threshold value can be any value. The reliability of pattern determining processing (step S503) is higher for a higher predetermined threshold value. For example, when the degree of similarity calculated in step S703 has a value of 0 to 1.0, the predetermined threshold value can be, for example, a value in the range of 0.4 to 0.9, such as 0.6. If it is judged in step S704 that the degree of similarity calculated in step S703 does not exceed a predetermined threshold value, the processing proceeds to step S705.

In step S705, the variable i is incremented. Steps S703 to S705 are repeated until it is judged that the degree of similarity exceeds the predetermined threshold value in step S704.

If it is judged that the degree of similarity calculated in step S703 exceeds the predetermined threshold value in step S704, the processing proceeds to step S706 and ends. This is because the specific pattern contained in an output of the processing means 221 can be determined to be a pattern of a motion indicated by the ith supervisory pattern whose degree of similarity has been judged to exceed the predetermined threshold value.

If none of the degrees of similarity calculated in step S703 is determined to exceed the predetermined threshold value in step S704, a determination of “no motion” can be made to end the processing.

The example discussed above has explained an example of learning a supervisory pattern obtained from a healthy individual, but the present invention is not limited thereto. For example, a supervisory pattern to be learned can be a pattern contained in a processed myoelectric signal obtained from a myoelectric signal obtained from a patient with the same level or milder impairment than the subject, or a pattern contained in a processed myoelectric signal obtained from the subject himself/herself. This enables the determination means 222 to conduct evaluation that matches the movement level of the subject, leading to increased motivation of the subject. For example, comparing the subject's pattern with a pattern of a patient having a milder impairment than the subject and showing the result thereof clarifies the goal the subject should strive for, thus increasing the motivation of the subject.

In the example discussed above, the degree of similarity with each supervisory pattern was sequentially calculated until it was judged that the degree of similarity exceeded a predetermined threshold value, but the present invention is not limited thereto. For example, calculation of all of the degrees of similarity with each supervisory pattern together by using a neural network is also within the scope of the present invention.

For example, the calculation means of the determination means 222 can be configured to calculate a degree of similarity between an output of the processing means 221 (processed myoelectric signal) and each supervisory pattern using a neural network in step S702′ instead of step S702. In this regard, the neural network can be, for example, a multilayer perceptron having an input layer, a hidden layer, and an output layer. The output layer of the multilayer perceptron can comprise the same number of output nodes as the number of motions to be identified. The hidden layer of the multilayer perceptron can comprise any number of nodes. The weighting coefficient for each node of the hidden layer of the multilayer perceptron can be calculated based on a combination of each supervisory pattern and a teaching signal indicating what motion each supervisory pattern is in the learning processing in step S701. For example, the weighting coefficient for each node can be calculated so that a value of the output layer when a supervisory pattern is inputted into the input layer would be a value of an teaching signal corresponding to the supervisory pattern. This is performed, for example, by backpropagation. Each node of a neural network output layer whose weighting coefficient has been calculated in this manner becomes associated with the motion corresponding to each supervisory pattern.

For example, if the weighting coefficient for each node is calculated using a combination of a first supervisory pattern and an teaching signal (1.0, 0.0, 0.0, 0.0, 0.0, 0.0, 0.0, 0.0) indicating that the motion is a mouth opening and closing motion of a jaw movement, a combination of a second supervisory pattern and an teaching signal (0.0, 1.0, 0.0, 0.0, 0.0, 0.0, 0.0, 0.0) indicating that the motion is a tapping motion of a jaw movement, a combination of a third supervisory pattern and an teaching signal (0.0, 0.0, 1.0, 0.0, 0.0, 0.0, 0.0, 0.0) indicating that the motion is a tongue raising motion of a tongue movement, a combination of a fourth supervisory pattern and an teaching signal (0.0, 0.0, 0.0, 1.0, 0.0, 0.0, 0.0, 0.0) indicating that the motion is a right side translation motion of a tongue movement, a combination of a fifth supervisory pattern and an teaching signal (0.0, 0.0, 0.0, 0.0, 1.0, 0.0, 0.0, 0.0) indicating that the motion is a left side translation motion of a tongue movement, a combination of a sixth supervisory pattern and an teaching signal (0.0, 0.0, 0.0, 0.0, 0.0, 1.0, 0.0, 0.0) indicating that the motion is a forward extension motion of a tongue movement, a combination of a seventh supervisory pattern and an teaching signal (0.0, 0.0, 0.0, 0.0, 0.0, 0.0, 1.0, 0.0) indicating that the motion is a left and right movement motion of a tongue movement, and a combination of an eighth supervisory pattern and an teaching signal (0.0, 0.0, 0.0, 0.0, 0.0, 0.0, 0.0, 1.0) indicating that the motion is a dry swallowing motion of a throat movement when distinguishing 8 motions shown in Table 1 using a neural network having 8 output nodes, the first node of the output layer of the neural network is associated with a mouth opening and closing motion of a jaw movement, the second node is associated with a tapping motion of a jaw movement, the third node is associated with a tongue raising motion of a tongue movement, the fourth node is associated with a right side translation motion of a tongue movement, the fifth node is associated with a left side translation motion of a tongue movement, the sixth node is associated with a forward extension motion of a tongue movement, the seventh node is associated with a left and right movement motion of a tongue movement, and the eighth node is associated with a dry swallowing motion of a throat movement. Examples of ideal outputs of a neural network with weighting coefficient for each node calculated in this manner include an output of 1.0 by the first node of the output layer and an output of 0 by the other nodes when a processed myoelectric signal obtained from a myoelectric signal from a mouth opening and closing motion of a jaw movement is inputted. However, an ideal output is actually hardly ever obtained due to the effect of noise or the like that coexists with a myoelectric signal. In actuality, one or more nodes of the output layer would output a value in the range of 0 to 1. The value of each node of the output layer corresponds to the degree of similarity between a processed myoelectric signal that has been inputted and a supervisory pattern corresponding to a respective motion to which each node is associated.

For example, an output of (0.0, 0.2, 0.0, 0.8, 0.0, 0.0, 0.0, 0.0) indicates that a pattern contained in a processed myoelectric signal that has been inputted is slightly similar to a supervisory pattern corresponding to a tapping motion of a jaw movement associated with the second node, and more similar to a supervisory pattern corresponding to a right side translation motion of a tongue movement associated with the fourth node, and is not similar to supervisory patterns corresponding to motions associated with other nodes. In this regard, if the predetermined threshold value is for example 0.5, a pattern contained in a processed myoelectric signal that has been inputted is determined to be a pattern of a right side translation motion of a tongue movement.

For example, an output of (0.0, 0.0, 0.4, 0.5, 0.0, 0.7, 0.0, 0.4) indicates that the pattern contained in a processed myoelectric signal which has been inputted is slightly similar to supervisory patterns corresponding to a tongue raising motion of a tongue movement, a right side translation motion of a tongue movement, and a dry swallowing motion associated with the third, fourth, and eighth nodes, respectively, and is similar to a supervisory pattern corresponding to a forward extension motion of a tongue movement associated with the sixth node, and is not similar to supervisory patterns corresponding to movements associated with other nodes. In this regard, if the predetermined threshold value is for example 0.5, a pattern contained in a processed myoelectric signal that has been inputted is determined as a pattern of a right side translation motion of a tongue movement and a forward extension motion of a tongue movement.

In a preferred embodiment, processing in step S503 is processing for determining which one or more of a pattern of a motion of a jaw movement, a pattern of a motion of a tongue movement, and a pattern of a motion of a throat movement correspond to a pattern contained in a processed myoelectric signal. A pattern of a motion of a jaw movement comprises a pattern of a mouth opening and closing motion, a pattern of a motion of a tongue movement comprises a pattern of a tongue raising motion, a pattern of a right side translation motion of the tongue, a pattern of a left side translation motion of the tongue, and a pattern of a forward extension motion of the tongue, and a pattern of a motion of a throat movement comprises a pattern of a dry swallowing motion. Identification of these patterns enables diagnosis of whether a subject has a swallowing impairment or not or diagnosis of a motion level in a swallowing motion. In a more preferred embodiment, processing in step S503 also identifies a pattern of a tapping motion in a jaw movement, and a pattern of a left and right movement motion of the tongue in a tongue movement in addition to the patterns of motions discussed above. This can improve the precision of the diagnosis of whether a subject has a swallowing impairment or not or diagnosis of a motion level in a swallowing motion.

In step S701 in a preferred embodiment, the learning means of the determination means 222 learns a supervisory pattern of a motion of a jaw movement including a supervisory pattern of a mouth opening and closing motion, supervisory patterns of motions of a tongue movement including a supervisory pattern of a tongue raising motion, a supervisory pattern of a right side translation motion of the tongue, a supervisory pattern of a left side translation motion of the tongue, and a supervisory pattern of a forward extension motion of the tongue, and a supervisory pattern of a motion of a throat movement including a supervisory pattern of a dry swallowing motion. The determination means 222 learns, for example, characteristics of a supervisory pattern of a motion of a jaw movement, a supervisory pattern of a motion of a tongue movement, and a supervisory pattern of a motion of a throat movement received via the interface unit 210, and stores the characteristics in the database unit 250. The characteristics of a supervisory pattern of a motion of a jaw movement, a supervisory pattern of a motion of a tongue movement, and a supervisory pattern of a motion of a throat movement can comprise, for example, a characteristic of a signal intensity distribution, a characteristic of a frequency distribution, and a characteristic of time series, as explained in Table 1. It can be identified whether a muscle closest to a position where measurement electrodes are fitted is active with a characteristic of a signal intensity distribution. A difference in the primary active muscle can be identified with a characteristic of a frequency distribution. A temporal change in the frequency and intensity of a myoelectric signal can be identified with a characteristic of time series. A pattern of a motion of a jaw movement, a pattern of a motion of a tongue movement, and a pattern of a motion of a throat movement can be clearly distinguished with a combination of these characteristics. A characteristic of a supervisory pattern of a motion of a jaw movement, a supervisory pattern of a motion of a tongue movement, and a supervisory pattern of a motion of a throat movement can also comprise, for example, a characteristic of a signal waveform. In this regard, learning means can learn a supervisory pattern for no motion being performed in addition to the aforementioned supervisory patterns.

In step S702 in a preferred embodiment, variable i is defined as i=1.

In step S703 in a preferred embodiment, calculation means of the determination means 222 calculates the degree of similarity between a pattern contained in an output of the processing means 221 after applying processing in step S502 (processed myoelectric signal) and the ith supervisory pattern. In this regard, the ith supervisory pattern is one of the supervisory patterns learned in step S701. For example, the first supervisory pattern is a supervisory pattern of a mouth opening and closing motion of a jaw movement, the second supervisory pattern is a supervisory pattern of a tongue raising motion of a tongue movement, the third supervisory pattern is a supervisory pattern of a right side translation motion of a tongue movement, the fourth supervisory pattern is a supervisory pattern of a left side translation motion of a tongue movement, the fifth supervisory pattern is a supervisory pattern of a forward extension motion of a tongue movement, and the sixth supervisory pattern is a supervisory pattern of a dry swallowing motion of a throat movement. The calculation means can calculate the degree of similarity between an output of the processing means 221 (processed myoelectric signal) and the ith supervisory pattern by using a known similarity calculating technique such as normalized cross-correlation.

In step S704 in a preferred embodiment, it is judged whether the degree of similarity calculated in step S703 exceeds a predetermined threshold value. The predetermined threshold value can be any value. The reliability of pattern determining processing (step S503) is higher for a higher predetermined threshold value. For example, when the degree of similarity calculated in step S703 has a value of 0 to 1.0, the predetermined threshold value can be, for example, a value in the range of 0.4 to 0.9, such as 0.6. If it is judged in step S704 that the degree of similarity calculated in step S703 does not exceed a predetermined threshold value, the processing proceeds to step S705.

In step S705 in a preferred embodiment, the variable i is incremented. Steps S703 to S705 are repeated until it is judged that the degree of similarity exceeds the predetermined threshold value in step S704.

If it is judged that the degree of similarity calculated in step S703 exceeds the predetermined threshold value in step S704 in a preferred embodiment, the processing proceeds to step S706 and ends. This is because the specific pattern contained in an output of the processing means 221 can be determined to be a pattern of a motion indicated by the ith supervisory pattern whose degree of similarity has been judged to exceed the predetermined threshold value.

In the preferred embodiment discussed above, the calculation means of the determination means 222 can be configured to calculate a degree of similarity between an output of the processing means 221 (processed myoelectric signal) and each supervisory pattern using a neural network in step S702′ instead of step S702.

The above examples have explained that the learning means of the determination means 222 learns in step S701 of step S503, but the learning means of the determination means 222 may learn in advance at least two of a supervisory pattern of a motion of a jaw movement, a supervisory pattern of a motion of a tongue movement, and a supervisory pattern of a motion of a throat movement by the processing similar to that in step S701, before starting processing 500. In such a case, step S701 is omitted in the processing in step S503. Even when the calculation means of the determination means 222 calculates a degree of similarity between an output of the processing means 221 (processed myoelectric signal) and each supervisory pattern using a neural network in step S702′ instead of step S702, the learning means of the determination means 222 can similarly learn a supervisory pattern of a motion to be identified using a neural network before starting processing 500.

The learning processing comprises, for example, detecting, by a myoelectric device, a myoelectric signal indicating the activity of a muscle when a subject without an impairment in a predetermined movement comprising at least two of a jaw movement, a tongue movement, and a throat movement (i.e., healthy individual) performs a predetermined movement, receiving a detected myoelectric signal by the processor unit 220, outputting, by the processor unit 220, a processed myoelectric signal by applying predetermined processing on the received myoelectric signal, and learning, by the processor unit 220, a pattern contained in the processed myoelectric signal as a pattern in a predetermined movement. The predetermined processing is, for example, the same processing as the processing by the processing means 221 discussed above.

Learning processing can be performed by using, for example, a supervisory pattern of a motion of a jaw movement, a supervisory pattern of a motion of a tongue movement, and a supervisory pattern of a motion of a throat movement, preferably a supervisory pattern of a mouth opening and closing motion, a supervisory pattern of a tongue raising motion, a supervisory pattern of a right side translation motion of the tongue, a supervisory pattern of a left side translation motion of the tongue, a supervisory pattern of a forward extension motion of the tongue, and a supervisory pattern of a dry swallowing motion. The determination means 222 subjected to learning processing using these patterns can be used as a classifier, which can identify a pattern of a mouth opening closing motion, a pattern of a tongue raising motion, a pattern of a right side translation motion of the tongue, a pattern of a left side translation motion of the tongue, a pattern of a forward extension motion of the tongue, and a pattern of a dry swallowing motion that are useful as an indicator in diagnosis of a swallowing impairment. More preferably, this can be performed using a supervisory pattern of a mouth opening and closing motion, a supervisory pattern of a tapping motion, a supervisory pattern of a tongue raising motion, a supervisory pattern of a right side translation motion of the tongue, a supervisory pattern of a left side translation motion of the tongue, a supervisory pattern of a forward extension motion of the tongue, a supervisory pattern of a left and right translation motion of the tongue, and a supervisory pattern of a dry swallowing motion. The determination means 222 subjected to learning processing using these patterns can be used as a classifier, which can identify a pattern of a mouth opening closing motion, a pattern of a tapping motion, a pattern of a tongue raising motion, a pattern of a right side translation motion of the tongue, a pattern of a left side translation motion of the tongue, a pattern of a forward extension motion of the tongue, a pattern of a left and right translation motion of the tongue, and a pattern of a dry swallowing motion that are further useful as an indicator in diagnosis of a swallowing impairment.

FIG. 7B depicts an example of processing in step S513 (processing performed by determination means 222 for determining a motion level in a jaw movement, tongue movement, or throat movement of a subject based on a pattern contained in a processed myoelectric signal).

In step S711, the learning means of the determination means 222 learns a supervisory pattern of a motion corresponding to a predetermined motion among a supervisory pattern of a motion of a jaw movement, a supervisory pattern of a motion of a tongue movement, and a supervisory pattern of a motion of a throat movement. For example, the determination means 222 learns a supervisory pattern of a motion of a jaw movement, a supervisory pattern of a motion of a tongue movement, or a supervisory pattern of a motion of a throat movement received through the interface unit 210, and stores the pattern in the database unit 250. The supervisory pattern is a pattern contained in a processed myoelectric signal obtained by applying the same processing as the processing by the processing means 221 discussed above on a myoelectric signal obtained from a healthy individual performing a motion of a jaw movement, a motion of a tongue movement, or a motion of throat movement. The characteristics of a supervisory pattern of a motion of a jaw movement, a supervisory pattern of a motion of a tongue movement, and a supervisory pattern of a motion of a throat movement can comprise, for example, a characteristic of a signal intensity distribution, a characteristic of a frequency distribution, and a characteristic of time series, as explained in Table 1. It can be identified whether a muscle closest to a position where measurement electrodes are fitted is active with a characteristic of a signal intensity distribution. A difference in the primary active muscle can be identified with a characteristic of a frequency distribution. A temporal change in the frequency and intensity of a myoelectric signal can be identified with a characteristic of time series. A pattern of a motion of a jaw movement, a pattern of a motion of a tongue movement, and a pattern of a motion of a throat movement can be clearly distinguished with a combination of these characteristics. A characteristic of a supervisory pattern of a motion of a jaw movement, a supervisory pattern of a motion of a tongue movement, and a supervisory pattern of a motion of a throat movement can also comprise, for example, a characteristic of a signal waveform.

In step S712, the calculation means of the determination means 222 can calculate a degree of similarity between a pattern contained in an output of the processing means 221 after applying the processing in step S512 (processed myoelectric signal) and a supervisory pattern. The calculation means can calculate a degree of similarity between an output of the processing means 221 (processed myoelectric signal) and a supervisory pattern by using a known similarity calculating technique such as normalized cross-correlation. Alternatively, the calculation means can be configured to calculate a degree of similarity between an output of the processing means 221 (processed myoelectric signal) and a supervisory pattern using the neural network discussed above.

The degree of similarity discussed above can be utilized, for example, for determining a motion level of a subject by the determination means 222. For example, when the degree of similarity is represented as 0 to 1.0, a degree of similarity of 1.0 indicates that a motion level is the same level as that of a healthy individual. A degree of similarity at or below a predetermined threshold value indicates that a subject is unable to perform the motion. A degree of similarity greater than a predetermined threshold value but less than 1.0 indicates that a subject is able to perform the motion, but not as high as that of a healthy individual. If for example the predetermined threshold value is 0.5, a degree of similarity of 0.6 indicates that the motion level is about 20% compared to a motion level of a healthy individual, a degree of similarity of 0.7 indicates that the motion level is about 40% compared to a motion level of a healthy individual, a degree of similarity of 0.8 indicates that the motion level is about 60% compared to a motion level of a healthy individual, and a degree of similarity of 0.9 indicates that the motion level is about 80% compared to a motion level of a healthy individual.

If, for example, a subject is asked to perform a dry swallowing motion of a throat movement to determine a degree of similarity between a pattern of a motion contained in a myoelectric signal obtained therefrom and a supervisory pattern of dry swallowing, a degree of similarity of 1.0 indicates that a dry swallowing motion level is the same level as that of a healthy individual. A degree of similarity at or below a predetermined threshold value indicates that a subject is unable to perform a dry swallowing motion. A degree of similarity that is greater than a predetermined threshold value but less than 1.0 indicates that a subject is able to perform a dry swallowing motion, but has an impairment in a throat movement and a dry swallowing motion level is not as high as that of a healthy individual. If for example the predetermined threshold value is 0.5, a degree of similarity of 0.6 indicates that the motion level is about 20% compared to a dry swallowing motion level of a healthy individual, a degree of similarity of 0.7 indicates that the motion level is about 40% compared to a dry swallowing motion level of a healthy individual, a degree of similarity of 0.8 indicates that the motion level is about 60% compared to a dry swallowing motion level of a healthy individual, and a degree of similarity of 0.9 indicates that the motion level is about 80% compared to a dry swallowing motion level of a healthy individual.

A motion level based on a degree of similarity can be presented to a subject, for example, by displaying the level on a monitor as a numerical value or by expressing the movement with an avatar of the subject. This enables subjects to see their own impairment level relative to a healthy individual. Alternatively, a subject in rehabilitation can check the level of improvement in rehabilitation by having motion levels presented over time.

The degree of similarity discussed above can also be utilized, for example, for evaluating the presence or absence of an impairment in a movement of a subject. For example, if it is judged that a degree of similarity calculated in step S712 does not exceed a predetermined threshold value upon judgment of whether the degree of similarity calculated in step S712 exceeds the predetermined threshold value, the processing proceeds to step S714 and a predetermined movement of a subject is evaluated as having an impairment. If it is judged that a degree of similarity calculated in step S712 exceeds a predetermined threshold value, the processing proceeds to step S715 and a predetermined movement of a subject is evaluated as having no impairment. The predetermined threshold value can be any value. The reliability of processing for evaluating the presence of absence of an impairment (step S513) is higher for a higher predetermined threshold value. For example, when the degree of similarity calculated in step S712 has a value of 0 to 1.0, the predetermined threshold value can be, for example, a value in the range of 0.4 to 0.9, such as 0.7.

If for example a subject is asked to perform a dry swallowing motion of a throat movement to determine a degree of similarity between a pattern of a movement contained in a myoelectric signal obtained therefrom and a supervisory pattern of dry swallowing, a degree of similarity that is no more than 0.6, when a predetermined threshold value is 0.6, indicates that there is an impairment in a dry swallowing motion, and a degree of similarity that is greater than 0.6 indicates that there is no impairment in a dry swallowing motion.

The presence or absence of an impairment in a movement of a subject can be evaluated, for example, by asking the subject to perform a plurality of motions. For example, processing can be configured so that if, among the plurality of motions, the number of motions with a degree of similarity greater than a predetermined threshold value is less than a predetermined number, this indicates that there is an impairment in the movement in the subject, and if the number of motions with a degree of similarity greater than a predetermined threshold value is greater than a predetermined number, this indicates that there is no impairment in the movement in the subject.

If for example a subject is asked to perform a mouth opening and closing motion of the jaw, a tongue raising motion, a right side translation motion of the tongue, a left side translation motion of the tongue, a forward extension motion of the tongue, and a dry swallowing motion to determine the degrees of similarity between a pattern of motion contained in a myoelectric signal for each motion and each of a supervisory pattern of a mouth opening and closing motion of the jaw, a supervisory pattern of a tongue raising motion, a supervisory pattern of a right side translation motion of the tongue, a supervisory pattern of a left side translation motion of the tongue, a supervisory pattern of a forward extension motion of the tongue, and a supervisory pattern of a dry swallowing motion, the number of degrees of similarity that are greater than the predetermined threshold value among the degrees of similarity of the six motions of less than 4 indicates, when the predetermined number is 4, that there is an impairment in a swallowing movement or a chewing movement of the subject. A number of degrees of similarity that are greater than the predetermined threshold value among the degree of similarities of the six motions of 4 or greater indicates, when the predetermined number is 4, that there is no impairment in a swallowing movement or a chewing movement of the subject.

The above examples have explained that the learning means of the determination means 222 learns in step 711 of step S513, but the learning means of the determination means 222 may learn in advance a supervisory pattern of a motion corresponding to a predetermined motion among a supervisory pattern of a motion of a jaw movement, a supervisory pattern of a motion of a tongue movement, and a supervisory pattern of a motion of a throat movement by the same processing as step S711, before starting processing 500. In such a case, step S711 is omitted in the processing in step S513. Even when the calculation means of the determination means 222 calculates a degree of similarity between an output of the processing means 221 (processed myoelectric signal) and a supervisory pattern using a neural network in step S712, the learning means of the determination means 222 can similarly learn a supervisory pattern of a motion to be identified using a neural network before starting processing 500.

The learning processing comprises, for example, detecting, by a myoelectric device, a myoelectric signal indicating the activity of a muscle when a subject without an impairment in a predetermined movement comprising a jaw movement, a tongue movement, or a throat movement (i.e., healthy individual) performs a predetermined movement, receiving a detected myoelectric signal by the processor unit 220, outputting, by the processor unit 220, a processed myoelectric signal by applying predetermined processing on the received myoelectric signal, and learning, by the processor unit 220, a pattern contained in a processed myoelectric signal as a pattern in a predetermined movement. The predetermined processing is, for example, the same processing as the processing by the processing means 221 discussed above.

Learning processing can be performed by using, for example, a supervisory pattern of a motion of a jaw movement, a supervisory pattern of a motion of a tongue movement, and a supervisory pattern of a motion of a throat movement, preferably a supervisory pattern of a mouth opening and closing motion, a supervisory pattern of a tongue raising motion, a supervisory pattern of a right side translation motion of the tongue, a supervisory pattern of a left side translation motion of the tongue, a supervisory pattern of a forward extension motion of the tongue, and a supervisory pattern of a dry swallowing motion. The determination means 222 subjected to learning processing using these patterns can be used as a classifier, which can identify a pattern of a mouth opening closing motion, a pattern of a tongue raising motion, a pattern of a right side translation motion of the tongue, a pattern of a left side translation motion of the tongue, a pattern of a forward extension motion of the tongue, and a pattern of a dry swallowing motion that are useful as an indicator in diagnosis of a swallowing impairment. More preferably, this can be performed using a supervisory pattern of a mouth opening and closing motion, a supervisory pattern of a tapping motion, a supervisory pattern of a tongue raising motion, a supervisory pattern of a right side translation motion of the tongue, a supervisory pattern of a left side translation motion of the tongue, a supervisory pattern of a forward extension motion of the tongue, a supervisory pattern of a left and right translation motion of the tongue, and a supervisory pattern of a dry swallowing motion. The determination means 222 subjected to learning processing using these patterns can be used as a classifier, which can identify a pattern of a mouth opening closing motion, a pattern of a tapping motion, a pattern of a tongue raising motion, a pattern of a right side translation motion of the tongue, a pattern of a left side translation motion of the tongue, a pattern of a forward extension motion of the tongue, a pattern of a left and right translation motion of the tongue, and a pattern of a dry swallowing motion that are further useful as an indicator in diagnosis of a swallowing disorder.

While one supervisory pattern with a degree of similarity exceeding a predetermined threshold value was determined in an example depicted in FIG. 7A, determination of a plurality of supervisory patterns with a degree of similarity exceeding a predetermined threshold value is also within the scope of the present invention. For example, the processing can be configured so that step S703 to step S704 are performed on all learned supervisory patterns to determine one or more supervisory patterns judged to have a degree of similarity exceeding a predetermined threshold value, and determine each of the plurality of patterns contained in a processed myoelectric signal is a pattern of motion indicated by each of the determined one or more supervisory patterns. For example in step S702′, the processing can be configured to determine one or more supervisory patterns judged to have a degree of similarity exceeding a predetermined threshold value from an output of a neural network and determine that each of the plurality of patterns contained in a processed myoelectric signal is a pattern of a motion indicated by each of one or more of the determined supervisory patterns. For example, the processing can be configured so that step S703 to step S704 are performed on all learned supervisory patterns to determine one optimal supervisory pattern from one or more supervisory patterns judged to have a degree of similarity exceeding a predetermined threshold value. For example in step S702′, the processing can be configured to determine an optimal supervisory pattern from one or more supervisory patterns judged to have a degree of similarity exceeding a predetermined threshold value from an output of a neural network. An optimal supervisory pattern can be determined, for example, by determining a supervisory pattern with the highest degree of similarity. An optimal supervisory pattern can be determined, for example, by comparison with past data. For example, the tendency of a motion is estimated from results recognized in the most recent recognition processing to determine a supervisory pattern of a motion in alignment with the tendency as an optimal supervisory pattern. For example, when a degree of similarity is judged to exceed a predetermined threshold value for a supervisory pattern of a tongue raising motion and a supervisory pattern of a forward extension motion after a pattern of a tongue raising motion has been recognized in the three most recent recognition processing, a supervisory pattern of a tongue raising motion is determined as an optimal supervisory pattern from the result of the most recent recognition processing. While an optimal supervisory pattern can be determined by any approach, the tendency of a motion is preferably estimated from a result recognized in the most recent recognition processing to determine a supervisory pattern of a motion in alignment with the tendency as an optimal supervisory pattern, because an error in determining patterns can be compensated by reference to past data.

The present invention is not limited to the aforementioned embodiments. It is understood that the scope of the present invention should be interpreted solely by the scope of the claims. It is understood that those skilled in the art can implement an equivalent scope, based on the descriptions of the invention and common general knowledge, from the descriptions of the specific preferred embodiments of the invention.

### INDUSTRIAL APPLICABILITY

The present invention has utility as an invention providing a system, method, and program for recognizing myoelectric signal-originating motions comprising at least two of a motion of a jaw movement, a motion of a tongue movement, and a motion of a throat movement.

### REFERENCE SIGNS LIST

10 System
100 Myoelectric device
111, 112 Myoelectric sensor
113, 113′ Pair of measurement electrodes
114, 114′ Pair of measurement electrodes
115 Transmission unit 115
200 Computer apparatus
210 Interface unit
220 Processor unit
230 Memory unit
250 Database unit

1. A system for evaluating a movement of a subject, the system comprising:
detection means for detecting a myoelectric signal indicating an activity of a muscle when a subject performs a movement intended to be a predetermined movement comprising a jaw movement, a tongue movement, and a throat movement;
processing means for outputting a processed myoelectric signal by applying predetermined processing on the detected myoelectric signal; and
determination means for determining a motion level in the predetermined movement of the subject, based on a pattern contained in the processed myoelectric signal.

2.-18. (canceled)

19. The system of claim 1, wherein the determination means is subjected to learning processing using a supervisory pattern of a motion corresponding to the predetermined movement among a supervisory pattern of a motion of a jaw movement, a supervisory pattern of a motion of a tongue movement, and a supervisory pattern of a motion of a throat movement, so that a degree of similarity to a myoelectric signal generated when there is no impairment in the predetermined movement is outputted,
wherein the determination means comprises:
calculation means for calculating a degree of similarity between the pattern contained in the processed myoelectric signal and the supervisory pattern.

20. The system of claim 19, wherein the learning processing is applied using a supervisory pattern of a motion of a jaw movement, a supervisory pattern of a motion of a tongue movement, and a supervisory pattern of a motion of a throat movement.

21. The system of claim 20, wherein the learning processing is applied using at least a supervisory pattern of a mouth opening and closing motion associated with the motion of a jaw movement, at least a supervisory pattern of a tongue raising motion, a supervisory pattern of a right side translation motion of the tongue, a supervisory pattern of a left side translation motion of the tongue, and a supervisory pattern of a forward extension motion of the tongue associated with the motion of a tongue movement, and at least a supervisory pattern of a dry swallowing motion associated with the motion of a throat movement.

22. The system of claim 21, wherein the learning processing is applied by further using a supervisory pattern of a tapping motion associated with the motion of a jaw movement and a supervisory pattern of a left and right translation motion of the tongue associated with the motion of a tongue movement.

23. The system of claim 21, wherein the predetermined processing comprises frequency analysis processing, and the processed myoelectric signal comprises time series data for a frequency distribution of the myoelectric signal.

24. The system of claim 23, wherein the frequency analysis processing comprises a short-time FFT.

25. The system of claim 23, wherein the predetermined processing further comprises signal intensity extraction processing, and the processed myoelectric signal further comprises time series data for signal intensities of the myoelectric signal.

26. The system of claim 19, wherein the determination means further evaluates a presence or absence of an impairment in the predetermined movement based on the degree of similarity.

27. The system of claim 1,
wherein the detection means comprises a myoelectric sensor comprising two pairs of measurement electrodes, and
wherein the two pairs of measurement electrodes are configured to be able to detect a myoelectric signal indicating an activity of a jaw muscle, a tongue muscle, or a throat muscle.

28. A method for evaluating a movement of a subject, the method comprising:
detecting a myoelectric signal indicating an activity of a muscle when a subject performs a movement intended to be a predetermined movement comprising a jaw movement, a tongue movement, and a throat movement;
outputting a processed myoelectric signal by applying predetermined processing on the detected myoelectric signal; and
determining a motion level in the predetermined movement of the subject, based on a pattern contained in the processed myoelectric signal.

29. A classifier used for evaluating a movement of a subject, the classifier comprising:
detection means for detecting a myoelectric signal indicating an activity of a muscle when a subject with no impairment in a predetermined movement comprising a jaw movement, a tongue movement, and a throat movement performs the predetermined movement;
processing means for outputting a processed myoelectric signal by applying predetermined processing on the detected myoelectric signal; and
learning means for learning a pattern contained in the processed myoelectric signal as a pattern in the predetermined movement.

30. The classifier of claim 29, wherein the predetermined movement comprises at least a mouth opening and closing motion associated with a motion of the jaw movement, at least a tongue raising motion, a right side translation motion of the tongue, a left side translation motion of the tongue, and a forward extension motion of the tongue associated with a motion of the tongue movement, and at least a dry swallowing motion associated with a motion of the throat movement.

31. The classifier of claim 30, wherein the predetermined movement further comprises a tapping motion associated with a motion of the jaw movement and a left and right translation motion of the tongue associated with a motion of the tongue movement.
